(19) United States
(12) Patent Application Publication
(10) Pub. No.: US 2025/0257189 A1
(43) Pub. Date: Aug. 14, 2025

(54) PROCESS FOR DEPOLYMERIZING POLYURETHANE

(71) Applicants: AARHUS UNIVERSITET, Aarhus C (DK); TEKNOLOGISK INSTITUT, Aarhus C (DK)

(72) Inventors: Martin Bundgaard JOHANSEN, Aarhus C (DK); Bjarke Skyum DONSLUND, Aarhus C (DK); Steffan Kvist KRISTENSEN, Aarhus C (DK); Anders Thyboe LINDHARDT, Viby J (DK); Troels SKRYDSTRUP, Aarhus C (DK)

(73) Assignees: AARHUS UNIVERSITET, Aarhus C (DK); TEKNOLOGISK INSTITUT, Aarhus C (DK)

(21) Appl. No.: 18/854,862

(22) PCT Filed: Apr. 5, 2023
(86) PCT No.: PCT/EP2023/058993

§ 371 Date: Oct. 7, 2024

(30) Foreign Application Priority Data
Apr. 7, 2022 (EP) 22167190.2

(51) Int. Cl. C08J 11/24; C07C 209/62; C07C 209/86; C08G 18/08; C08G 18/48

(52) U.S. Cl. CPC C08J 11/24; C07C 209/62; C07C 209/86; C08G 18/08; C08G 18/4829; C08J 2375/04

(57) ABSTRACT

The present invention relates to a method for production of a mixture comprising polyol(s) and amine(s) from a polyurethane, said method comprising the steps of: a) providing a starting material comprising the polyurethane, b) suspending the starting material comprising the polyurethane in a suspending agent comprising an alcohol to provide a polyurethane suspension, c) heating the polyurethane suspension to a temperature in the range of 150° C. to 400° C. to provide said mixture comprising polyol(s) and amine(s), wherein said alcohol is a C₄-C₈ tertiary alcohol. Another aspect involves the use of an alcohol selected from the group consisting of a C₄-C₁₂ tertiary alcohol and a C₃-C₁₀ secondary alcohol for catalysing the conversion of a polyurethane to provide polyol(s) and amine(s).

### TECHNICAL FIELD OF THE INVENTION

The present invention relates to recycling of polyurethane using a chemical process to depolymerize polyurethane material in order to obtain the original monomers of the polyurethane i.e. polyols and isocyanates. In particular the present invention relates to a novel process for converting polyurethane into polyols and amines, respectively, where the amines may subsequently be converted to isocyanate monomers. The monomers or monomer precursors are obtained by reaction of polyurethane with particular alcohols and optionally with a base at elevated temperatures that provide high yields and facile separation and isolation of said monomers or precursors thereof.

### BACKGROUND OF THE INVENTION

Polymers and engineered plastic products have become an integrated part of our society with a worldwide production exceeding 360 million tons in 2019. One important and versatile class of plastic is polyurethane (PU) that constitutes of repeating carbamate functional groups formulated from a polyol and a diisocyanate. PU can be found in everyday consumer goods such as furniture, insulation material in cooling devices and in constructions, shoe components, and kitchen sponges to name a few but is also found in advanced materials such as aviation and auto-motive parts. As the PU precursors are traditionally prepared from fossil resources, a circular PU economy is therefore highly desirable. Besides cutting of the need for new raw, fossil materials, recycling would ideally mean zero landfilling and significantly reduce the necessity to incinerate highly engineered materials. The cross-linking between the monomeric units in many PU materials (thermoset plastics) results in the absence of a melting point and there-fore makes mechanical reuse by melting and remoulding physically impossible.

PU can be mechanically reused by granulating and reformulating the powder as a filler in combination with a binder (typically polyisocyanates) and external compression to produce second generation products. Although this rebinding offers an avenue for a range of padding products from PU foam scrap and production line slabstock trim, the material will in most cases be of a different quality than the prime product and have a narrow product line. In addition, flexible foam scrap can only be rebonded a finite number of times before the properties can no longer meet minimum specification. Lastly, the European market for PU trim is already saturated, evident by the yearly export of 300,000 tons annually, and is therefore unlikely to absorb future post-consumer PU trim, if collected. Thus, it is desirable to develop processes that affords the original monomeric PU building blocks, namely polyol and diisocyanate or its industrial precursor dianiline that would enable repolymerization of the components into new first generation products.

A handful of chemical recycling processes such as hydrolysis, acidolysis, aminolysis, alcoholysis, hydrogenation, and glycolysis exist to progress towards such recycling. Glycolysis has received most attention on industrial scale. Glycolysis can be subcategorized into a method called split-phase glycolysis and traditional glycolysis.

Split-phase glycolysis stems from the two-phase post-reaction mixture that comprises an upper phase rich on polyol and a lower phase containing mainly the glycolysis solvent (e.g. glycol) and dianilines, plus dicarbamates and aminocarbamates derived of the latter.

WO2019219814 discloses such a split-phase reaction where flexible PU foam is treated with base, bismuth catalyst, 2-pyrrolidone co-catalyst, and e.g. a number of polyvalent alcohols as solvent at elevated temperatures (Examples E1-E6). The upper phase was then further purified by heating the solution in additional diglycerol and subsequent centrifuging to give 99 wt % pure polyol. The lower phase could be hydrolysed at 200° C. under basic conditions 24 hours to afford mainly dianiline in water (examples 10-11). Thus, split-phase glycolysis targets original polyol, while further reactions and refinement have to be performed to isolate the dianiline including separating it from water.

WO2020260387 discloses a glycolysis reaction of polyurethane followed by the post-reaction addition of secondary solvent (such as toluene) to induce splitting of the phases (abstract). Thus, current glycolysis technologies strive to separate polyol and dianiline by additional steps of separation and purification to optimize the yield of carbon-based material, especially targeting the polyol.

In contrast, the traditional glycolysis process provides a single-phase reaction mixture from the heating of PU foam with catalyst in glycol. The resulting mixture comprises residual glycol, polyol, dianiline, and (amino) carbamate derivatives of the dianiline. As amines alters the kinetics and controllability of the foaming process in addition to changing the foam composition and properties, they are unwanted in the recycled polyol.

US2002010222A1 therefore discloses capping of amines with e.g. an epoxide or a cyclic carbonate to convert the amine functional groups into non-virgin polyols (paragraph and examples). The mixture obtained from this additional process primarily contains a range of polyols with a measurable content of aromatics and carbonyl functionalities different from the virgin polyol. Therefore, significant amounts of virgin polyol is still required in addition to the recycled polyol to reformulate PU foams with acceptable physical properties. Thus, single-phase glycolysis followed by amine capping affords a recycled polyol that is chemically and physically different from the virgin polyol and the dianiline is lost from a recycling perspective, as it is incorporated in the recycled polyol.

Hence, an improved method of converting polyurethanes and closely related polymers back to its monomer components or precursors thereof would be advantageous, and in particular a more high-yielding and facile method for producing polyol monomers and isocyanate monomer precursors, in the form of amines, from polyurethane would be advantageous.

### SUMMARY OF THE INVENTION

Thus, an object of the present invention relates to a method of producing polyol(s) and amine(s) from a polyurethane wherein the polyols correspond to the polyol monomer of the polyurethane and where in the amines correspond to the amine precursor of the isocyanate monomers of the polyurethane, to enable conversion of end-of-life polyurethane back to its pristine monomers.

In particular, it is an object of the present invention to provide a method that solves the above mentioned problems of the prior art with isolating and/or separating the reaction products of e.g. glycolysis or hydrolysis of polyurethane from the reaction solvents and to achieve sufficient yields to allow for facile isolation of polyol fraction and particularly the amine fraction from the mixture obtained from the solvolysis process of the present invention.

Thus, a first aspect of the present invention relates to a method for production of a mixture comprising polyol(s) and amine(s) from a polyurethane, said method comprising the steps of:
a) providing a starting material comprising the polyurethane,
b) suspending the starting material comprising the polyurethane in a suspending agent comprising an alcohol to provide a polyurethane suspension,
c) heating the polyurethane suspension to a temperature in the range of 150° C. to 400° C. to provide said mixture comprising polyol(s) and amine(s),
   wherein said alcohol is selected from the group consisting of a C₄-C₁₂ tertiary alcohol, and a C₃-C₁₀ secondary alcohol.

A second aspect of the present invention relates to a use of an alcohol selected from the group consisting of a C₄-C₁₂ tertiary alcohol and a C₃-C₁₀ secondary alcohol in the conversion of a polyurethane to provide polyol(s) and amine(s).

### BRIEF DESCRIPTION OF THE FIGURES

FIG. 1 shows ¹H and ¹³C NMR spectra of the MDA fraction in the flexible solid PU part of a shoe shank as provided in example 2.

FIG. 2 shows ¹H and ¹³C NMR spectra of the polyol fraction in the flexible solid PU part of a shoe shank as provided in example 2.

FIG. 3 shows ¹H and ¹³C NMR spectra of the MDA fraction in a generic solid thermoplastic (rigid polyurethane) as provided in example 2.

FIG. 4 shows ¹H and ¹³C NMR spectra of the polyol fraction in a generic solid thermoplastic (rigid polyurethane) as provided in example 2.

FIG. 5 shows ¹H and ¹³C NMR spectra of the MDA fraction in a PU textile as 5 provided in example 3.

FIG. 6 shows ¹H and ¹³C NMR spectra of the polyol fraction in a PU textile as provided in example 3.

FIG. 7 shows infrared spectroscopy of the removal of the polyurethane from the textile fabric in example 3. The major peaks in elastane fiber (PU), for example around 1100 cm⁻¹ are shown to be removed in the leftover fabric.

FIG. 8 illustrates the workflow of the method of the present invention including isolating polyol and amine by HCl salt precipitation (see Example 4 for description of setups). First, 2 g of flexible shredded foam mattress was provided (top picture). Second, the shredded foam mattress was subjected to the method of the present invention (Step A, see example 4 for details). Third, the content of the reactor was transferred to a round-bottomed flask and concentrated under reduced pressure (Step B, middle picture). The amines were precipitated as salts using HCl in CPME (Step C). The resulting suspension was allowed to stir at room temperature, filtered, and washed with excess CPME (Step D). The precipitated dianiline was dried in vacuo to afford 2HCl·TDA as a beige solid. The filtrate was concentrated under reduced pressure and dried in vacou to afford polyol as a yellow oil, resulting in a 92 wt % and 91 wt % recovery in a glovebox and bench setup, respectively. The final products are shown in the bottom picture.

FIG. 9 shows a comparison of the ¹H NMR spectrum of the lower phase of a split-phases alcoholysis reaction (cf. example 6) prior to the method of the present invention (top spectrum) and the NMR spectrum of the reaction mixture after alcoholysis of the lower phase according to the present invention (bottom spectrum).

The present invention will now be described in more detail in the following.

### DETAILED DESCRIPTION OF THE INVENTION

### Definitions

Prior to discussing the present invention in further details, the following terms and conventions will first be defined:

### Polyurethane

In the present context, the term “polyurethane” is defined in the broadest sense as any polymer comprising alternating organic units joined by carbamate (urethane) bonds. Particularly polyurethanes in this context are polymers stemming from polyol (often diol) and isocyanate (often diisocyanate) monomers. Polyurethanes may be in the form of flexible foams, flexible solids, rigid foams, or rigid solids depending on monomer types, crosslinking and other variations in structure. Polyurethanes also include non-linear polymers such as e.g. poly (urethane-isocyanurate) and polyurethanes making up parts of block-co-polymers comprising polyurethanes. Partly degraded polyurethanes are also included, such as polyurethane fragments or by-products of incomplete degradation processes. This may particularly include fragments/by-products in the so-called “lower phase” or dicarbamate/amine-carbamate phase resulting from split-phase alcoholysis as described in e.g. WO2019219814.

### Starting Material Comprising the Polyurethane

In the present context, the term “starting material comprising the polyurethane” relates to any material comprising an amount of polyurethane and possibly other components that are not detrimental to the method of the present invention. Such other component or materials may be other solid materials that are inert to the present process or are otherwise easily separated from the products of the present process. Therefore, in principle, there is no lower limit to the percentage of polyurethane present in the starting material, as long as the remainder of material does not interfere with the method of the present invention, and is separable from the resulting polyols and amines. Such starting materials may for example include textiles or fibers comprising polyurethanes, and particularly it may include the dicarbamate/amine-carbamate phase or “lower phase” of split-phase alcoholysis of polyurethanes, i.e. the remaining phase when the polyol phase has been removed in split-phase glycolysis, as described in e.g. WO2019219814.

### Polyol(s)

In the present context, the term “polyol(s)” relates to the polyol(s) obtained from the method of the present invention which preferably correspond to a large extend to the original polyol monomer of the polyurethane. These are often diols, such as e.g. alkylene diols (HO—(CH₂)ₙ—OH), but may also be triols or higher polyols. The polyurethane may comprise more than one kind of polyol monomer, for example in block-co-polymers, and hence the term “polyol(s)” is used herein.

### Amine(s)

In the present context, the term “amine(s)” relates to amines that are amine precursors of the isocyanate monomers of the polyurethane of the invention. As for polyols, the amines are polyamines, and most often diamines. The amines are often positioned on an aromatic group, and are thus often variations of di-anilines. Thus one example of a common amine in the present context is toluenediamines (TDA), which may be in the form of either 2,4- or 2,6-diaminotoluene. Thus, TDAs are precursors of toluenediisocyanates (TDI). The polyurethane may comprise more than one kind of isocyanate monomer, for example in block-co-polymers, or in the form of regio-isomers, and hence the term “amine(s)” is used herein to describe the isocyanate precursors obtained.

### Mixture Comprising Polyol(s) and Amine(s)

In the present context, the term “mixture comprising polyol(s) and amine(s)” relates to the mixture obtained after subjecting a polyurethane to the method of the present invention. This crude mixture primarily comprises the polyols and amines as defined above and depending on conditions also residual solvent, catalyst and reactants, and also possible minor by-products and/or impurities. Unreacted parts of the starting material as defined above may also be present.

### Suspending Agent

In the present context, the term “suspending agent” may be used interchangeably with “suspending composition” or simply “composition”. The terms relate to the composition or agent in which the polyurethane polymer is suspended prior to adding optional further reactants and heating to facilitate the solvolysis reaction. The suspending agent may thus simply be the alcohol of the invention as defined herein, or may optionally comprise further solvents or additives. Typically, only the polymeric polyurethane will be suspended in the suspending agent, while other components and the reaction products will be dissolved in the suspending agent.

### Alcohol

In the present context, the term “alcohol” relates in the broadest sense to organic alcohols, i.e. organic molecules comprising at least one hydroxyl (—OH) group. In this context, the alcohol is one capable of transforming into a water molecule and an alkene, preferably via an elimination reaction and preferably under the condition of the method of the present invention. Such as alcohols are preferably low molecular weight, volatile organic alcohols, which also function well as a solvent and is easily removed after reaction completion.

### Suspension

In the present context, the term “suspension” relates to a liquid comprising an undissolved element. In this case this particularly means the suspending agent as defined herein, which comprises suspended polyurethane. During the method of the present invention, the polyurethane will be in suspension until it is sufficiently degraded into smaller monomers or oligomers which dissolve.

### Heating

In the present context, the term “heating” relates to applying a particular temperature to the suspension and/or solution of the present method. Usually heating will be applied starting at ambient temperature, typically room temperature and until a target temperature is reached where the temperature is kept constant until the reaction of the invention (depolymerisation/solvolysis) has completed to a suitable extent. Heating may be performed at constant pressure or at constant volume, e.g. in an autoclave type setup. The duration of heating in the present context relates to the duration at the target temperature.

### Polyol Fraction

In the present context, the term “polyol fraction” relates to the fraction obtainable from the method of the present invention wherein the polyol(s) resulting from the depolymerisation of the polyurethane are present. The polyol fraction consists essentially of polyol(s). Minor amounts of impurities, solvent and/or reactant residuals may also be present.

### Amine Fraction

In the present context, the term “amine fraction” relates to the fraction obtainable from the method of the present invention wherein the amine(s) resulting from the depolymerisation of the polyurethane are present. The amine fraction consists essentially of amine(s), such as e.g. di-anilines. Minor amounts of impurities, solvent and/or reactant residuals may also be present.

### Additional Solvents

In the present context, the term “additional solvents” relates to any industrially applicable solvent other than the alcohol of the invention already present. Such as co-solvent or additional solvent may be added to improve solubility of reactants or products or to reduce the amount of alcohol needed in the initial suspension of polyurethane. As for the alcohol of the invention the additional solvent should be easily removed after the reaction has occurred, e.g. via work-up or concentration under reduced pressure.

### Mono-Valent Alcohol

In the present context, the term “mono-valent alcohol” relates to organic molecules comprising a single hydroxyl-functional group (—OH). Other functional groups may be present, but only one hydroxyl-group is present per molecule.

### Base

In the present context, the term “base” relates to compounds that may form hydroxide ions in aqueous solutions, i.e. compounds that are able to deprotonate a water molecule. In the present context bases may particularly be Brønsted bases. Examples of useful bases in the present context are inorganic salts of hydroxide(s) (OH—), such as NaOH, KOH, Ca(OH)₂, and carboxy acids such as salts or metal conjugates of carboxylic acids, including e.g. NaOAc, KOAc and dibutyltindilaurate (DBTDL).

### Aqueous-Organic Extraction

In the present context, the term “aqueous-organic extraction” relates to the work-up method wherein compounds are separated by their solubility in water and an organic solvent respectively, possibly over several extraction steps. The organic solvent is immiscible in water.

### Hydroxyl Value

In the present context, the term “hydroxyl value” relates to the number of milligrams of potassium hydroxide required to neutralize the acetic acid taken up on acetylation of one gram of a chemical substance that contains free hydroxyl groups. Hydroxyl value is a measure of the content of free hydroxyl groups in a chemical substance, usually expressed in units of the mass of potassium hydroxide (KOH) in milligrams equivalent to the hydroxyl content of one gram of the chemical substance. The analytical method used to determine hydroxyl value traditionally involves acetylation of the free hydroxyl groups of the substance with acetic anhydride in pyridine solvent. After completion of the reaction, water is added, and the remaining unreacted acetic anhydride is converted to acetic acid and measured by titration with potassium hydroxide.

The hydroxyl value can be calculated using the following equation. Note that a chemical substance may also have a measurable acid value affecting the measured endpoint of the titration. The acid value (AV) of the substance, determined in a separate experiment, enters into this equation as a correction factor in the calculation of the hydroxyl value (HV):

$$HV = [ [ ( 56.1 ) ( N ) ( {V}_{B} - {V}_{acet} ) ] / {W}_{acet} ] + AV$$

Where HV is the hydroxyl value;
V_{B} is the amount (ml) potassium hydroxide solution required for the titration of the blank;
V_{acet} is the amount (ml) of potassium hydroxide solution required for the titration of the acetylated sample;
W_{acet} is the weight of the sample (in grams) used for acetylation;
N is the normality of the titrant;
56.1 is the molecular weight of potassium hydroxide;
AV is a separately determined acid value of the chemical substance.

The present inventors have surprisingly found that the certain organic alcohols, as described herein, and which would not normally be contemplated for an alcoholysis reaction of polyurethane, work extremely well in producing polyols and amines from polyurethanes.

Thus, a first aspect of the present invention relates to a method for production of a mixture comprising polyol(s) and amine(s) from a polyurethane, said method comprising the steps of:
a) providing a starting material comprising the polyurethane,
b) suspending the starting material comprising the polyurethane in a suspending agent comprising an alcohol to provide a polyurethane suspension,
c) heating the polyurethane suspension to a temperature in the range of 150° C. to 400° C. to provide said mixture comprising polyol(s) and amine(s),
   wherein said alcohol is selected from the group consisting of a C₄-C₁₂ tertiary alcohol, and a C₃-C₁₀ secondary alcohol.

Without being bound to theory it is believed that the alcohol of the present invention provides the required amount of water molecules via elimination of water from said alcohol to effectively facilitate a hydrolysis without the presence of a large excess of water, which may challenging to separate from the reaction products upon isolation of these.

The mixture comprising polyols and amines obtained from the method of the present invention may be separated into fractions comprising the polyols and the amines by known methods as described further below, and thus an embodiment of the present invention relates to the method as described herein, wherein the method comprises an additional step after step c) of
d) separating said polyol(s) from said amine(s) to provide a polyol fraction and an amine fraction.

The polyol fraction may optionally be further purified or used directly as pristine polyol monomer in the formation of new polyurethanes. The amine fraction may likewise be further purified and converted to e.g. isocyanate monomers, which may also be used as pristine monomers of polyurethanes. Amines are readily converted to isocyanates by known methods, including, but not limited to, e.g. phosgenation.

In an alternative embodiment, the method of the present invention has the primary goal of removing polyurethane from another material, which can then be essentially separated from the comprised polyurethane. One example could be a textile product (e.g. cotton) comprising e.g. elastane (a polyurethane). Steps a)-c) of the present invention can effectively remove the polyurethane from the textile without degrading the other textile materials.

Therefore in an alternative embodiment the present invention relates to the method as described herein, wherein the method comprises an additional step after step c) of
c′) separating said mixture comprising polyol(s) and amine(s) from said starting material to provide said starting material free of polyurethane.

In these embodiments, the starting material comprising a polyurethane may preferably be a textile. The polyurethane is thus separated from said starting material by conversion to a mixture comprising polyols and amines followed by washing away these compounds with a suitable solvent or liquid. The starting material is thus isolated from any polyurethane originally comprised therein.

The obtained mixture of polyols and amines may be subjected to step d) with or after step c′), or if e.g. the amount of polyurethane is relatively small, the mixture may be discarded or used otherwise.

The suspending agent of the present invention may simply consist of the alcohol of the present invention, but may also comprise further solvents to e.g. aid in the solvation of products or reduce the amount of alcohol needed. Thus in one embodiment of the present invention the suspending agent comprises the alcohol and one or more additional solvents. The additional solvent is preferably an industrially applicable solvent. The additional solvent preferably has a boiling point in the range of 10-300° C., such as 20-250° C., such as 50-200° C., such as 60-180° C., such as preferably 80-150° C. An embodiment of the present invention relates to the method as described herein, wherein the additional solvent is selected from the list consisting of toluene, mixed xylenes, 2-Me-THF, and ethyl acetate, or any mixture thereof. Other reactants and catalysts may be added to the suspending agent, but are not considered part of the suspending agent, which rather consists of the alcohol and any additional solvents. An embodiment of the present invention relates to the method as described herein, wherein the suspending agent consists of the alcohol.

The alcohol of the present invention is preferably a solvent-like small molecule organic alcohol, with the claimed ability to produce a molecule of water via an elimination reaction. Preferably the alcohol is relatively low boiling to enable removal of excess alcohol after the reaction has occurred. Several features of an organic alcohol may improve its ability to produce a water molecule and facilitate the present reaction, including but not limited to being a tertiary or secondary alcohol, or to comprise functional groups that will result in conjugated or even aromatic double bonds upon elimination of water.

Therefore, an embodiment of the present invention relates to the method as described herein, wherein the alcohol is selected from the group consisting of a C₄-C₁₂ tertiary alcohol, and a C₃-C₁₀ secondary alcohol. More preferably, the alcohol is a C₄-C₁₀ tertiary alcohol, most preferably a C₄-C₈ tertiary alcohol.

An embodiment of the present invention relates to the method as described herein, wherein the alcohol is selected from the group consisting of tert-amyl alcohol, 1,1-dimethylallyl alcohol, tert-butanol, iso-propanol, methyl vinyl carbinol, and terpineol.

More preferably the alcohol is selected from the group consisting of tert-amyl alcohol, 1,1-dimethylallyl alcohol, tert-butanol, and terpineol. The most preferred alcohol is tert-amyl alcohol.

Further preferred attributes of the alcohol of the present invention include that the molecular weight of the alcohol is in the range of 50 g/mol to 200 g/mol, such as 55 g/mol to 160 g/mol, preferably 60 g/mol to 100 g/mol. The alcohol may preferably be a mono-valent alcohol. Further, the alcohol preferably has a boiling point in the range of 40° C. to 230° C., such as 50° C. to 150° C., such as 60-130° C., such as 70-120° C., such as preferably 90-110° C.

A further embodiment of the present invention relates to the method as described herein, wherein the alcohol is present in molar excess to the carbamate bonds in said polyurethane. Preferably the alcohol is the suspending agent for the polyurethane and a solvent for the polyol(s) and amine(s). An embodiment of the present invention relates to the method as described herein, wherein the alcohol is the only suspending agent for the polyurethane and the only solvent for the polyol(s) and amine(s).

As provided above the starting material comprising the polyurethane may simply be a polyurethane but may also comprise other materials as long as these are not detrimental to the production of polyols and amines of the present invention. The other materials may either be incorporated in the polyurethane polymer matrix (e.g. in a block-copolymer comprising a polyurethane) or may be a material which is simply not easily separable from the polyurethane (e.g. a fiber material or polymer mixture comprising a polyurethane).

Thus, an embodiment of the present invention relates to the method as described herein, wherein the starting material comprising a polyurethane is selected from the group consisting of a polyurethane, a poly (urethane-isocyanurate), a cross-linked polyurethane, a block co-polymer comprising polyurethane, a polymer mixture comprising a polyurethane, or any mixture thereof. Preferably, the starting material comprising a polyurethane comprises polyurethane in the range of 1-100% by weight, such as 50-100% by weight, such as 70-100% by weight, such as 80-100% by weight, such as 90-99% by weight, such as most preferably 90-95% by weight. An embodiment of the present invention relates to the method as described herein, wherein the starting material comprising a polyurethane is a polyurethane.

The fundamental idea of the present invention is to recycle polyurethane in the most sustainable way possible, i.e. by converting used polyurethane into its original monomers in high yields, to enable the manufacture of recycled polyurethane of high quality. Therefore, an embodiment of the present invention relates to the method as described herein, wherein the polyurethane is selected from the list consisting of an end-of-life polyurethane, a scrap polyurethane, and a production waste polyurethane.

The degradation of polyurethane into its original monomers or precursors thereof, i.e. polyols and amines may be improved by the addition of e.g. bases, additives and/or catalysts. Such additional reactants may optimise reaction conditions and facilitate lowering of reaction duration/heating (step c)), lowering the temperature of step c) or providing higher yields or a cleaner reaction.

The present inventors found that adding a base to the reaction increases the efficiency and turnover to monomeric products. Thus, one embodiment of the present invention relates to the method as described herein, wherein a base is further added to the polyurethane suspension. Preferably the base is added under step b), i.e. prior to step c). The base is preferably an inorganic base or a carboxylate base. Said base may preferably be selected from the group consisting of potassium hydroxide (KOH), sodium hydroxide (NaOH), dibutyltindilaurate (DBTDL), potassium acetate (KOAc), sodium acetate (NaOAc), sodium octanoate, potassium octanoate, tin dioctanoate, sodium hydride (NaH), K₃PO₄, and bismuth (III) neodecanoate. An embodiment of the present invention relates to the method as described herein, wherein said base is potassium hydroxide (KOH).

The present inventors also found that particularly for rigid polyurethanes it may be advantageous to add a nucleophile to aid the reaction, and thus in one embodiment of the present invention relates to the method as described herein, wherein a further alcohol or an amine is added to the polyurethane suspension. Preferably said further alcohol or amine is a primary alcohol or amine. The further alcohol or amine may be selected form the group consisting of methanol, ethanol, n-propanol, and n-butanol, methylamine, ethyl amine, propyl amine, hydroxyethylamine, ammonia, preferably methanol.

The further alcohol or amine is preferably added in a small amount as compared to the alcohol of step b) which is the primary driver of the reaction. Thus in an embodiment of the present invention the molar ratio between the alcohol of step b) and the further alcohol or amine is in the range of 100:1 to 10:1, such as 80:1 to 20:1, such as 60:1 to 40:1.

The temperature applied in step c) of the present method determines the duration and the need for further additives, bases or catalyst in the reaction in order to arrive at an industrially applicable method. If the reaction is performed e.g. in a batch reactor at constant volume the temperature, along with any gases produced in the reaction, will also determine the pressure in the reaction vessel.

Thus, an embodiment of the present invention relates to the method as described herein, wherein the temperature in step c) is in the range of 150° C. to 380° C., such as 160° C. to 360° C., such as 170° C. to 340° C., such as 180° C. to 320° C., such as 190° C. to 300° C., such as preferably 195° C. to 280° C.

In another embodiment the pressure during step c) is in the range of 0.5-200 bar, such as 0.8-50 bar, such as 0.9-30 bar, such as 1-20 bar, such as preferably approximately 1 bar.

An embodiment of the present invention relates to the method as described herein, wherein step c) has a duration in the range of 0.1-10 h, such as 0.5-8 h, such as 1-6 h, such as 1.5-4 h, such as 1.7-3 h, such as preferably 2-6 h.

Another embodiment of the present invention relates to the method as described herein, wherein step c) is performed in an atmosphere selected from the group consisting of nitrogen, argon and ambient air, or any mixture thereof, preferably nitrogen. Yet another embodiment of the present invention relates to the method as described herein, wherein the polyurethane suspension of step c) is physically agitated, such as preferably stirred. The method of the present invention, and particularly step c) of the present invention may be performed in any type of industrially applicable reactor including but not limited to a batch reactor, such as e.g. an autoclave, or in a continuous flow reactor or a continuous flow reaction system.

The mixture obtained in step c) comprising polyol(s) and amine(s) may optionally be subject to separation of the polyols from the amines as provided in step d) described above. Such separation may be achieved by a variety of methods well known to the skilled individual. The separation is further facilitated by the high crude yields obtained by the present method and resulting low amounts of by-products.

In one embodiment of the present invention the separation of polyol(s) and amine(s) in step d) is performed by a method comprising aqueous-organic extraction. The solubility of the polyols and amines (which are most often aromatic amines, i.e. anilines) in water and various organic solvents may differ and this may be utilised in aqueous-organic extraction method where reaction products are divided between an aqueous phase and an organic phase. Repeated extraction and washing of the phases may result in high “crude” purities of the polyol fraction and amine fraction obtained.

In another embodiment the separation of polyol(s) and amines(s) in step d) is performed by a method comprising precipitating the amine(s) as a salt. The amines may be precipitated in the presence of the polyols, and the precipitation is typically achieved by adding an acid to provide a crystalline salt of the amines (ammonium type salts, e.g. NH₄⁺Cl⁻).

Thus in one embodiment the amines are precipitated as a salt by contacting said mixture comprising polyol(s) and amines(s) with an inorganic acid. The inorganic acid may preferably be selected from the group consisting of HCl and H₂SO₄. In a preferred embodiment the precipitation of the amine(s) as a salt is preceded by concentration of said polyol(s) and amines(s), optionally under reduced pressure, and addition of a precipitation solvent in conjunction with said inorganic acid. The precipitation solvent may be selected from the group consisting of cyclopentyl methyl ether (CPME), diethyl ether, ethyl acetate, chloroform, dichloromethane, toluene, or any mixture thereof.

As the polarity of the polyols and amines will typically also differ, one embodiment of the present invention relates to the method as described herein, wherein the separation of polyol(s) and amines(s) in step d) is performed by a method comprising column chromatography. Differences in boiling points may also be utilised and thus an embodiment of the present invention relates to the method as described herein, wherein the separation of polyol(s) and amines(s) in step d) is performed by a method comprising distillation.

As the aim of the present invention is to provide pristine monomers of the polyurethane starting material, a preferred embodiment of the present invention is an embodiment wherein said polyol(s) are polyol monomers of said polyurethane.

The mixture obtained in step c) and the polyol fraction obtained from step d) should contain as few impurities, by-products and solvent/reactant residuals as possible, and thus a preferred embodiment of the present invention relates to the method as described herein, wherein the polyol fraction obtained from step d) comprises at least 90 mol % polyol(s), such as at least 95 mol %, such at least 98 mol %, such as at least 99 mol %, such as preferably at least 99.5 mol % polyol(s).

One possible measure of how pure and how close to the pristine polyol monomer of the starting polyurethane the product polyol(s) are is the hydroxyl value as compared to the hydroxyl value of the pure monomer. Therefore, an embodiment of the present invention relates to the method as described herein, wherein the polyol fraction has a hydroxyl value which is no more than 10% above the hydroxyl value of the pure monomer, such as no more than 7%, 5%, 3%, such as preferably no more than 2% above the hydroxyl value of the pure monomer.

As mentioned herein, the amine(s) provided by the present method may be readily converted to the corresponding isocyanates via known methods, such as e.g. phosgenation. The isocyanates thus obtained would correspond to the original monomers of the polyurethane that is subject to the method of the present invention. Therefore, preferably said amine(s) are precursors of the isocyanate monomers of said polyurethane. Also said amine(s) or salts thereof may preferably be further converted to isocyanate(s), such as isocyanate monomers of said polyurethane.

The mixture obtained in step c) and the amine fraction obtained from step d) should contain as few impurities, by-products and solvent/reactant residuals as possible, and therefore one embodiment of the present invention relates to the method as described herein, wherein the amine fraction obtained from step d) comprises at least 90 mol % amine(s) or salts thereof, such as at least 95 mol %, such at least 98 mol %, such as at least 99 mol %, such as preferably at least 99.5 mol % amine(s) or salts thereof. The amine(s) may preferably be precipitated as a salt, preferably a hydrochloric acid salt.

In relation to step a) which involves the provision of the starting material comprising a polyurethane, it is preferred (if necessary) that the starting material is pre-treated to maximize the contact surface of the polyurethane with the solvents and possible reactants in the suspension. Typically this means that the starting material is in the form of a powder or fine-cut/shredded material. Thus, an embodiment of the present invention relates to the method as described herein, wherein the polyurethane starting material has been subject to pre-treatment. The pre-treatment may preferably be selected from the group consisting of shredding, pulverizing, grinding, milling, and cutting. It is noted however that some forms of polyurethane starting materials may not require pre-treatment, i.e. if they are already in powder form, or in relatively small pieces.

In relation to the fractions of polyol and amine as obtained in step d) of the present method these may be subject to further purification if required. Thus, an embodiment of the present invention relates to the method as described herein, wherein the polyol fraction and/or amine fraction obtained in step d) are subsequently further purified. Further purification may be performed using salt-formation, crystallisation, re-crystallisation, column chromatography, distillation, or any combination thereof.

An alternative aspect of the present invention relates to a method for production of a mixture comprising polyol(s) and amine(s) from a polyurethane, said method comprising the steps of:
a) providing a starting material comprising the polyurethane,
b) suspending the starting material comprising the polyurethane in a suspending agent comprising an alcohol to provide a polyurethane suspension,
c) heating the polyurethane suspension to a temperature in the range of 150° C. to 400° C. to provide said mixture comprising polyol(s) and amine(s),
   wherein said alcohol is an alcohol capable of transforming into a water molecule and an alkene by an elimination reaction at the temperature range of step c).

Another alternative aspect of the present invention relates to a method for production of a composition comprising amine(s) from a polyurethane, said method comprising the steps of:
a) providing a starting material comprising the dicarbamate/amine-carbamate phase from a split-phase alcoholysis of said polyurethane,
b) contacting the starting material with a solvent comprising an alcohol to provide a reaction mixture,
c) heating the reaction mixture to a temperature in the range of 150° C. to 400° C. to provide said composition comprising amine(s),
   wherein said alcohol is selected from the group consisting of a C₄-C₁₂ tertiary alcohol, and a C₃-C₁₀ secondary alcohol.

The present inventors have surprisingly found that certain alcohols are particularly useful in facilitating the conversion of polyurethane to its monomers or precursors thereof.

Thus, a further aspect of the present invention relates to the use of an alcohol selected from the group consisting of a C₄-C₁₂ tertiary alcohol and a C₃-C₁₀ secondary alcohol in the conversion of a polyurethane to provide polyol(s) and amine(s). Yet a further aspect of the present invention relates to the use of an alcohol selected from the group consisting of a C₄-C₁₂ tertiary alcohol and a C₃-C₁₀ secondary alcohol for catalysing the conversion of a polyurethane to provide polyol(s) and amine(s).

Preferably the polyurethane is suspended in a suspending agent comprising said alcohol to provide a suspension. The suspension may be heated to a temperature in the range of 150° C. to 400° C. to provide a mixture comprising polyol(s) and amine(s). Also said polyol(s) and amine(s) may be separated to provide a polyol fraction and an amine fraction.

It should be noted that embodiments and features described in the context of one of the aspects of the present invention also apply to the other aspects of the invention. All patent and non-patent references cited in the present application, are hereby incorporated by reference in their entirety.

The invention will now be described in further details in the following non-limiting examples.

### EXAMPLES

### Materials and Methods

Unless otherwise stated, all reactions were set-up in a glovebox under an atmosphere of argon in an oven-dried 30 mL PTFE inlay fitted into a 45 mL dry autoclave cell. The pressure vessels were heated in aluminum blocks specially designed to fit the vessels. All alcohols used in this study were purchased as ‘anhydrous’, if possible, and opened inside an argon-filled glovebox and were used without further purification. Analytical thin layer chromatography (TLC) was performed on silica coated aluminum plates (Merck Kieselgel 60 F254), which were visualized using UV and/or stained with KMnO4 (1.5 g KMnO4 in 1.25 mL NaOH and 200 mL H2O). Flash column chromatography was performed on the Interchim Puriflash XS520plus automated column system using Interchim Puriflash prepacked columns with 30 μm spherical silica. NMR experiments were performed on a Bruker Ascend 400 spectrometer, where ¹H NMR and ¹³C NMR were recorded at 400 MHz and 101 MHZ, respectively. Chemical shifts are reported in parts per million (ppm) relative to the residual solvent signal of the deuterated solvent. HRMS spectra were recorded on a Bruker Maxis Impact Spectrometer (TOF, ESI). GC experiments were performed using an Agilent 8890 GC System (Column: HP-5 5% Phenyl methyl siloxane, 30 m×250 μm×0.25 μm). Specifications on the method: Inlet: 170° C., Detector: FID 300° C.; Carrier gas: He. Flow 2 mL/min. Oven: 50° C., hold 0.5 min; 35° C./min to 300° C., hold 1.7 min. All calibration curves for determining GC yields were obtained by using standard solutions of the analytes (2,4′-methylenedianiline, 4,4′-methylenedianiline, 2,4-toluenediamine, 2,6-toluenediamine, MeOH) in EtOAc.

### “Small Autoclave”

45 mL pressure vessels made from T316 stainless steel alloy, designed to handle a temperature range: −10-300° C. The vessel head consists of a single inlet/outlet valve fitted for 5000 Multi Reactor System (Parr®), thermowell for thermocouple insertion, removable PTFE gasket, screw cap and six stainless steel compression bolts for sealing the contents of the reaction well. Unless otherwise noted, all reactions were set up using a 30 mL PTFE inlay along with a PTFE coated magnet. All connections were sealed using PTFE tape.

### “Large Autoclave”

Berghof BR-300 High Pressure Reactor with an internal volume of appr. 390 mL made of Stainless Steel 316Ti. A homemade heating block that is adaptable to common heating- and stirring plates was built.

### General Procedure A

To a PTFE inlay fitted with a stir bar was added Flexible Foam (Mattress) (250 mg), base/additive (0.04 mmol), and solvent (5 mL) in an argon-filled glovebox. The PTFE inlay was placed in an auto-clave reactor, which was sealed and fastened using a hex key. The reactor was placed in a pre-heated aluminum block at the desired temperature for the desired period of time at 800 rpm stirring rate. The reactor was then cooled to room temperature. The reactor was opened and the thermowell was washed with EtOAc. The content of the reactor was transferred to a round-bottomed flask or a vial in which mesitylene (50 μL) was added as an internal standard and the mass of mesitylene was noted. An aliquot of 250 μL was transferred to a vial and the mixture was quickly filtered through a small cotton and celite plug (3-5 mm of celite in a glass pipette) followed by additional EtOAc into a 2 mL vial suited for GC-MS. The sample was analyzed using the GC method described above. The GC yield of dianilines was determined using pre-made calibration curves.

### General Procedure B

To a PTFE inlay fitted with a stir bar was added PU sample (250 mg), KOH (1.9 mg, 0.033 mmol) or DBTDL (25.4 mg, 0.04 mmol), and tert-amyl alcohol (5 mL) in an argon-filled glovebox. The PTFE inlay was placed in an autoclave reactor, which was sealed and fastened using a hex key. The reactor was placed in a pre-heated aluminum block at 225° C. for 2.25 h at 800 rpm stirring rate. The reactor was then cooled to room temperature. The reactor was opened and the thermowell was washed with EtOAc. The content of the reactor was transferred washing with EtOAc to a round-bottomed flask or a vial in which mesitylene (50 μL) was previously added as an internal standard (exact mass of mesitylene was noted). An aliquot of 250 μL was transferred to a vial and the mixture was quickly filtered through a small cotton and celite plug (3-5 mm of celite in a glass pipette) followed by additional EtOAc into a 2 mL vial suited for GC-MS. The sample was analysed using the GC method described above. The GC yield of dianilines was determined using pre-made calibration curves.

The aliquot was returned to the main crude mixture and the mixture was purified by automated flash column chromatography using an EtOAc/heptane eluent system from 1:9 to pure EtOAc. Following elution of anilines the polyol fraction was collected by flushing the column with MeOH.

For samples containing polymeric MDA (polyMDA) in the polyol fraction, polyol was recovered by dissolving the polyol fraction obtained from chromatography in 1 M HCl/brine (1:1, 2 mL) and extracting the aqueous phase with CH₂Cl₂ (5×2 mL). The organic phases were combined, dried over anhydrous Na₂SO₄, filtered, concentrated, and dried in vacuo overnight to afford the polyol. For the recovery of polymeric MDA, the aqueous phase was basified with 4 M NaOH till pH of 10-11 and was extracted with CH₂Cl₂ (5×2 mL). The organic phases were combined, dried over anhydrous Na₂SO₄, filtered, concentrated, and dried in vacuo overnight to afford polymeric MDA.

### General Procedure C

To a PTFE inlay fitted with a stir bar was added PU sample (250 mg), KOH (1.9 mg, 0.033 mmol) or DBTDL (25.4 mg, 0.04 mmol), MeOH (100 μL), and tert-amyl alcohol (5 mL) in an argon-filled glovebox. The PTFE inlay was placed in an autoclave reactor, which was sealed and fastened using a hex key. The reactor was placed in a pre-heated aluminum block at 225° C. for 4.5 h at 800 rpm stirring rate. The reactor was then cooled to room temperature. The reactor was opened and the thermowell was washed with EtOAc. The content of the reactor was transferred washing with EtOAc to a round-bottomed flask or a vial in which mesitylene (50 μL) was previously added as an internal standard (exact mass of mesitylene was noted). An aliquot of 250 μL was transferred to a vial and the mixture was quickly filtered through a small cotton and celite plug (3-5 mm of celite in a glass pipette) followed by additional EtOAc into a 2 mL vial suited for GC-MS. The sample was analyzed using the GC method described above. The GC yield of dianilines was determined using pre-made calibration curves.

The aliquot was returned to the main crude mixture and the mixture was purified by automated flash column chromatography using an EtOAc/heptane eluent system from 1:9 to pure EtOAc. Following elution of anilines the polyol fraction was collected by flushing the column with MeOH.

For samples containing polymeric MDA (polyMDA)* in the polyol fraction, polyol was recovered by dissolving the polyol fraction obtained from chromatography in 1 M HCl/brine (1:1, 2 mL) and extracting the aqueous phase with CH₂Cl₂ (5×2 mL). The organic phases were combined, dried over anhydrous Na₂SO₄, filtered, concentrated, and dried in vacuo overnight to afford the polyol. For the recovery of polymeric MDA, the aqueous phase was basified with 4 M NaOH till pH of 10-11 and was extracted with CH₂Cl₂ (5×2 mL). The organic phases were combined, dried over anhydrous Na₂SO₄, filtered, concentrated, and dried in vacuo overnight to afford polymeric MDA.

*In some cases polymeric MDA with one additional aniline unit (n=2) was isolated from column chromatography while higher order polymeric MDA (n>1) was always isolated from the above-described acid/base extraction.

### Example 1—Deconstruction of PU with Variation in Parameters

The screening was performed in an autoclave fitted with a 30 mL PTFE inlay stirred at 200° C. for 18 hours and the yield of the regioisomeric mixture of TDA was determined by GC-FID analysis (Table 1). A background reaction of 20% product was observed when using THE as solvent (entry 1). The conversion might be explained by residual water in the autoclave system. Primary alcohol (n-BuOH) showed similar yields as the background reaction (entries 2). Surprisingly, when the tertiary alcohol tert-amyl alcohol was applied, the yield was 88% (entry 3).

TABLE 1
Solvolysis of Flexible Foam Sample

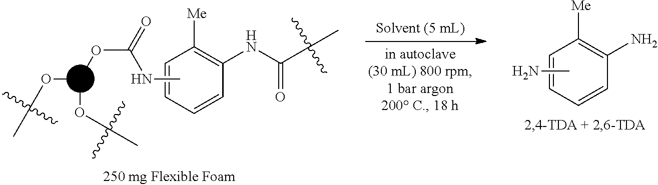

| Entry | Solvent | Yield^{[a]} |
|---|---|---|
| 1 | THF | 20% |
| 2 | n-BuOH | 17% |
| 3 | tert-Amyl alcohol | 88% |

^{[a]}GC-FID yield determined using 1,3,5-trimethylbenzene as internal standard.

The interesting effect of tert-amyl alcohol was further investigated by screening the reaction time, temperature, and addition of base (Table 2). Initially, the reaction time was reduced by half down to 2.25 h. Maintaining a reaction time of 2.25 h with an increased reaction temperature of 225° C. gave a 64% yield (entry 3). By elevating the temperature to 250° C., a yield of >95% was achieved in 2.25 h (entry 4). Simple inorganic bases such as NaOAc, KOAc, NaOH, and KOH were tested at 200° C. for 2.25 h (entries 5-8). The potassium salts performed better than the sodium salts with KOH affording the highest yield of 61%, which was increased to >95% at 225° C. (entry 9). Dibutyltin dilaurate (DBTDL) has been widely used as catalyst for the curing/foaming of PU and its performance in this deconstruction method stood out from the remaining base screening. Changing the solvent to 1 mL tert-amyl alcohol and 4 mL toluene significantly lowered the yield to 79% (entry 12). DBTDL proved more effective than KOH at 200° C. with a yield of 74% (entry 10) while it gave complete conversion to dianilines at 225° C. (entry 11). K₃PO₄ and Bi (III) neodecanoate could also be used with yields similar to NaOH (entries 13-14)

TABLE 2
Examples of Flexible Foam Deconstruction in tert-Amy Alcohol^{a}

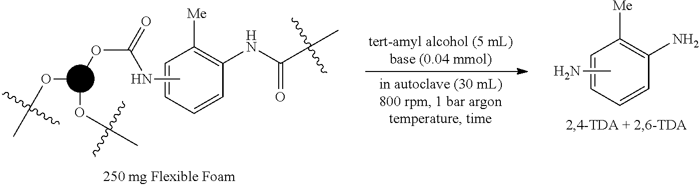

| Entry | Time | Temperature | Base | Yield^{[b]} |
|---|---|---|---|---|
| 1 | 18 h | 200° C. | — | 88% |
| 2 | 9 h | 200° C. | — | 59% |
| 3 | 2.25 h | 225° C. | — | 64% |
| 4 | 2.25 h | 250° C. | — | >95% |
| 5 | 2.25 h | 200° C. | NaOAc | 39% |
| 6 | 2.25 h | 200° C. | KOAc | 57% |
| 7 | 2.25 h | 200° C. | NaOH | 49% |
| 8 | 2.25 h | 200° C. | KOH | 61% |
| 9 | 2.25 h | 225° C. | KOH | >95% |
| 10 | 2.25 h | 200° C. | DBTDL | 74% |
| 11 | 2.25 h | 225° C. | DBTDL | >95% |
| 12 | 2.25 h | 225° C. | KOH | 79%^{c} |
| 13 | 2.25 h | 200° C. | K₃PO₄ | 47% |
| 14 | 2.25 h | 200° C. | Bi(III) | 45% |
| | | | neodecanoate | |

^{a}All reactions were performed in 45 mL autoclaves fitted with a 30 mL PTFE inlay at 800 rpm under one bar argon.
^{[b]}GC-FID yield of TDA was determined using 1,3,5-trimethylbenzene as an internal standard.
^{c}Solvent system: 4:1 solution of toluene and tert-amyl alcohol.
DBTDL: dibutyltin dilaurate.

The apparent unique role of tert-amyl alcohol using KOH as base was next compared to other low-molecular weight alcohols (Table 3). The tertiary alcohols proved to be the most effective with GC-FID yields of 78% and 72% for 1,1-dimethylallyl alcohol and tert-butanol, respectively. Anhydrous terpineol (available as an isomeric mixture) proved very effective but was not further investigated due to its unpractical high boiling point above 200° C. Methyl vinyl alcohol and i-propanol demonstrated that secondary alcohols had considerable conversion to the dianilines. Besides allylcarbinol with a yield of 71%, the primary (mono) alcohols were low yielding as evident from n-butanol and methanol.

TABLE 3
Effect of Using Tertiary, Secondary, and Primary Alcohols^{[a]}

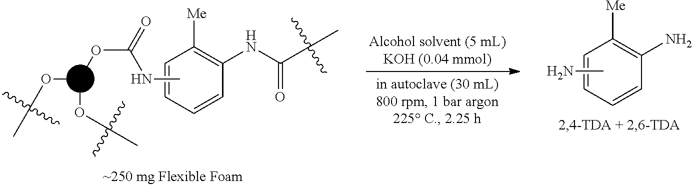

| | | | | |
|---|---|---|---|---|
| tert-amyl | 1,1- | tert-butanol | 60-85% α- | methyl vinyl |
| alcohol (TAA) | dimethylallyl | 72% | terpineol in | carbinol |
| 96% | alcohol | | isomeric | 62% |
| | 78% | | mixture | |
| | | | quant.^{b} (99%) | |
| i-propanol | n-butanol | Methanol | | |
| 65% | 31% | 18% | | |

^{[a]}GC-FID yield determined by using 1,3,5-trimethylbenzene as internal standard.
^{b}Water content of received anhydrous terpineol was determined to 2500 ppm by Karl-Fischer analysis.

### Example 2—Deconstruction of PU Samples

Having established conditions for the deconstruction of flexible PU foam systems, we set out to test a range of flexible PU materials including a foam mattress, a household kitchen sponge, an end-of-life car seat, rebonded granulate foam, and a pillow.

### Flexible Solid (Shoe Shank)

The PU was taken from a shoe shank.

The PU was shredded using a meat grinder.

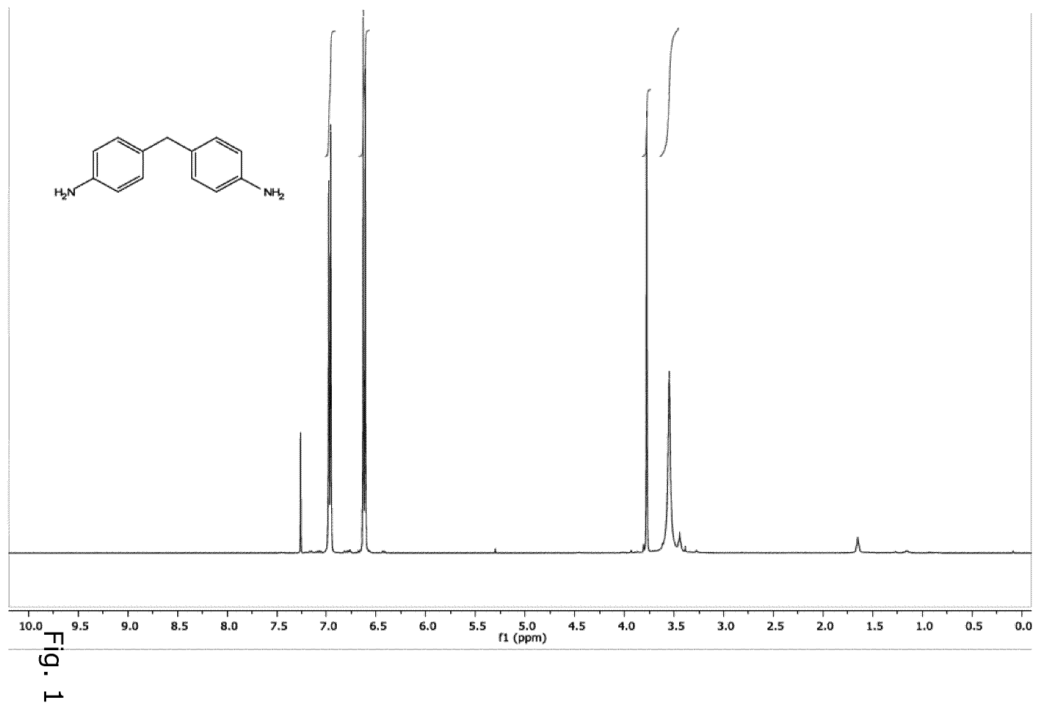

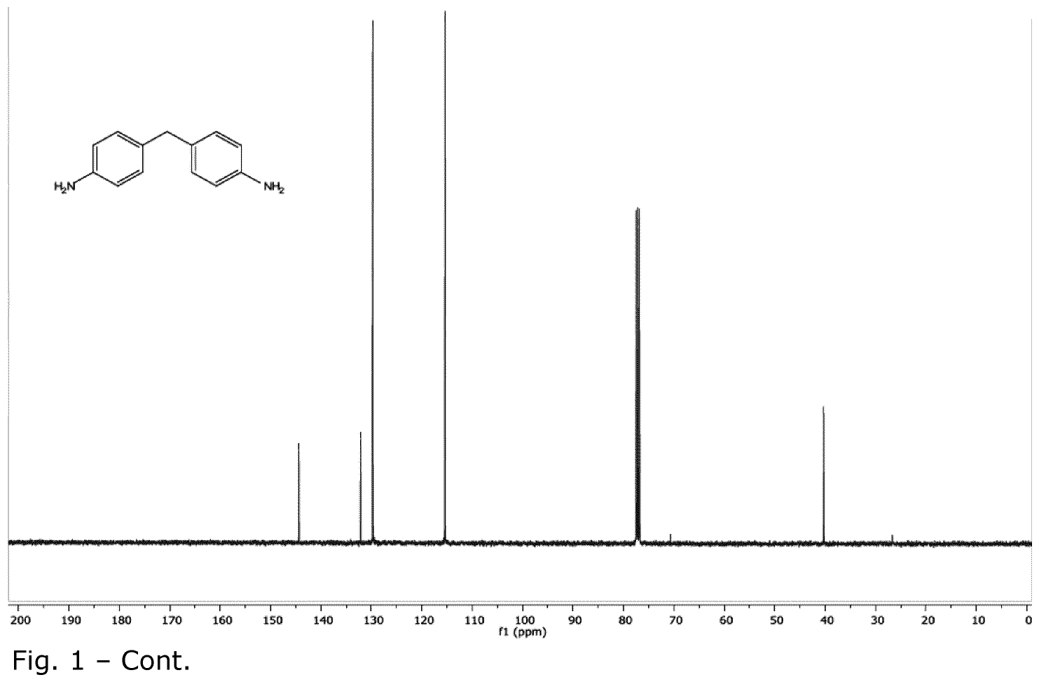

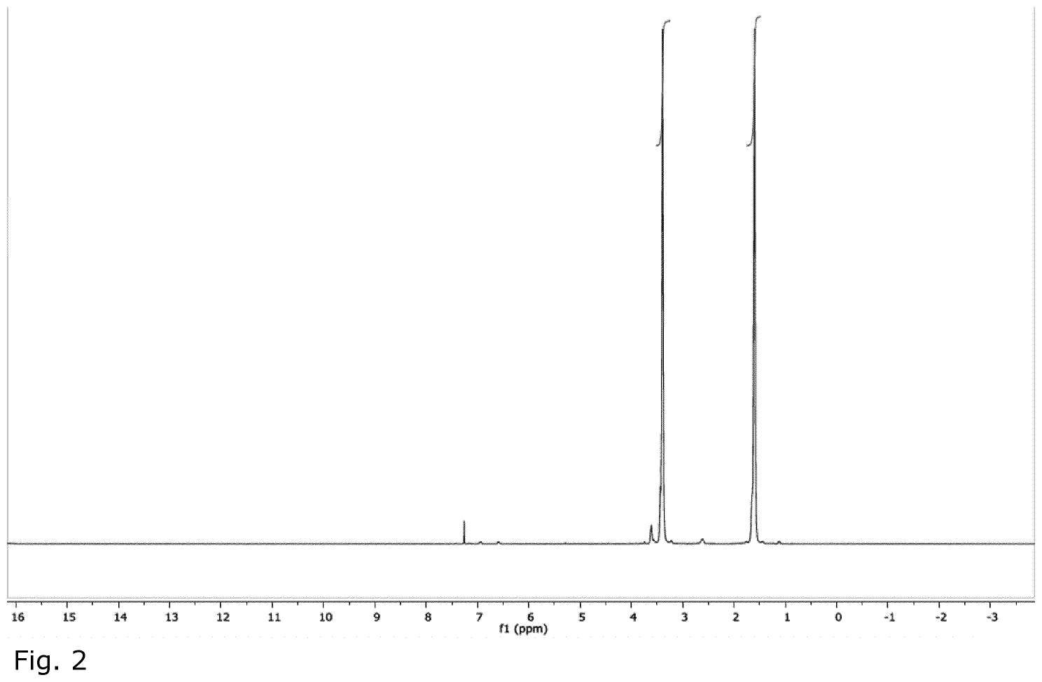

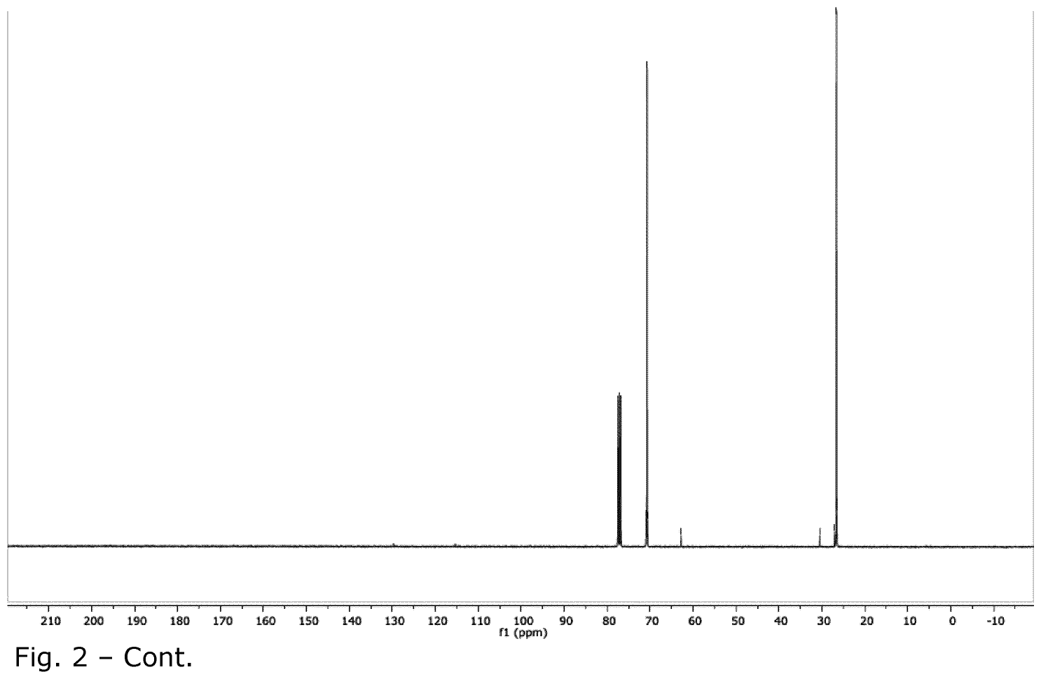

KOH as base. The PU (run 1: 248.5 mg; run 2: 250.8 mg) was deconstructed as described in General Procedure C to afford 4,4′-MDA (run 1: 33.1 mg; run 2: 30.8 mg, FIG. 1) as a yellow solid in an average yield of 32.0 mg and a polyol fraction (run 1: 162.6 mg; run 2: 178.2 mg, FIG. 2) as a yellow oil in an average yield of 170.4 mg.

### Flexible Foam (Mattress)

The flexible foam was formulated with the below indicated reagents:

| Flexible Foam (Mattress) | wt % |
|---|---|
| TDI | 41 |
| 3000 MW propoxylated glycerol | 100 |
| Water | 4 |
| Polysiloxane polyoxyalkylene block copolymer | 1 |
| (TEGOSTAB ® BF 2370) | |
| Stannous octoate | 0.2 |
| 33 LV: 33% triethylenediamine, 67% dipropylene glycol | 0.2 |

The foam was shredded on a modified twin screw extruder (Xinda, 65 mm twin screw extruder with 2000 mm barrel length) before chemical deconstruction.

KOH as base. The foam (run 1: 250.9 mg; run 2: 250.6 mg) was deconstructed as described in General Procedure B to afford TDA (run 1: 55.9 mg; run 2: 52.6 mg) as a brown solid in an average yield of 54.3 mg (2,4-:2:6-=4.1:1) and a polyol fraction (run 1: 185.1 mg; run 2: 169.9 mg) as a brown oil in an average yield of 177.5 mg.

DBTDL as base. The foam (run 1: 253.3 mg; run 2: 260.7 mg) was deconstructed as described in General Procedure B to afford TDA (run 1: 54.9 mg; run 2: 58.4 mg) as an off-white solid in an average yield of 56.7 mg (2,4-:2:6-=4.1:1) and a polyol fraction (run 1: 174.8 mg; run 2: 177.3 mg) as a brown oil in an average yield of 176.1 mg.

### Flexible Foam (Kitchen Sponge)

The sponge was acquired in Føtex (Danish supermarket, brand: Budget). The yellow flexible PU part of the sponge was chopped into smaller pieces using a scissor before chemical deconstruction.

KOH as base. The foam (run 1: 252.6 mg; run 2: 250.2 mg) was deconstructed as described in General Procedure B to afford TDA (run 1: 63.1 mg; run 2: 60.0 mg) as a yellow solid in an average yield of 61.6 mg (2,4-:2:6-=4.1:1) and a polyol fraction (run 1: 156.4 mg; run 2: 157.2 mg) as a yellow oil in an average yield of 156.8 mg.

### Flexible Foam (End-of-Life Car Seat)

The sponge was acquired from an end-of-life car seat from a Ford car.

A piece of the PU part of the seat was chopped into smaller pieces using a scissor before chemical deconstruction.

KOH as base. The foam (run 1: 254.2 mg; run 2: 249.8 mg) was deconstructed as described in General Procedure B to afford TDA (run 1: 44.4 mg; run 2: 42.6 mg) as a yellow solid in an average yield of 43.5 mg (2,4-:2:6-=4.3:1) and a polyol fraction (run 1: 80.6 mg; run 2: 108.2 mg) as a brown solid in an average yield of 94.4 mg.

### Rebonded Granulate Foam

The rebond foam was acquired on www.tm-materialer.dk (brand: Polytex). The foam was shredded using a Fritsch cutting mill fitted with a 1 mm filter. KOH as base. The foam (run 1: 258.0 mg; run 2: 254.4 mg) was deconstructed as described in General Procedure B to afford TDA (run 1: 34.1 mg; run 2: 37.2 mg) as a yellow solid in an average yield of 35.7 mg (2,4-:2:6-=4.4:1), MDA (run 1: 13.0 mg; run 2: 17.2 mg) as a yellow oil in an average yield of 15.1 mg (2,4′-:4,4′-=1:1.04), and a polyol fraction* (run 1: 160.4 mg; run 2: 157.1 mg) as a green solid in an average yield of 158.8 mg.

*The polyol fraction contained a considerable amount of insoluble solids that could originate from fire retardants or other solid additives.

### Viscoelastic Foam (Pillow)

The foam was a blend of flexible foam and viscoelastic foam found in a pillow (IKEA, Rosenskärm). The PU was shredded using a Fritsch cutting mill fitted with a 1 mm filter.

KOH as base. The PU (run 1: 250.6 mg; run 2: 251.7 mg) was deconstructed as described in General Procedure B to afford a fraction with TDA and MDA (run 1: 41.5 mg; run 2: 43.0 mg) as a yellow oil in an average yield of 42.3 mg (25.4 mg TDA, 2,4-:2,6-=4.3:1; 16.9 mg MDA 2,4′-:4:4′-=1:1.8), and a fraction with polyol and polymeric MDA (run 1: 141.7 mg; run 2: 126.8 mg) as a brown oil in an average

yield of 134.3 mg.

We investigated the deconstruction of an insulating rigid foam salvaged from a refrigerator (Table 4). Using the optimized conditions from the flexible foam, a 35.0 mg yield of 2,4-MDA and 4,4′-MDA (methylene diphenyl amine) was determined by GC-FID analysis (entry 1). The yield was not significantly increased by prolonging the reaction time to 4.5 h (entry 2). In addition, a precipitate was observed in the reaction mixtures, suggesting that the polymer was only partially deconstructed. The polymeric MDA (polyMDA) from the rigid foam was not quantified but our earlier report on this material gave 47.2 mg MDA and 73.9 mg polyMDA with a mass recovery of 80%. Repeating the first experiment in a solvent mixture of four parts tert-amyl alcohol and one part MeOH gave a homogenous reaction mixture but a low yield of 17.6 mg MDA. It was hypothesized that MeOH might act as small nucleophile breaking up the polymer into soluble oligomers by trans-carbamoylation reaction but some aromatic amines were capped as the corresponding O-methyl carbamate. Thus, the addition of MeOH was lowered to 100 UL and the reaction time set to 4.5 h which led to an increase in yield to 43.9 mg MDA (entry 4). A similar trend was seen when substituting KOH with DBTDL (entries 5-6).

TABLE 4
Investigation of Rigid Refrigerator Foam Deconstruction Mediated by tert-Amyl Alcohol

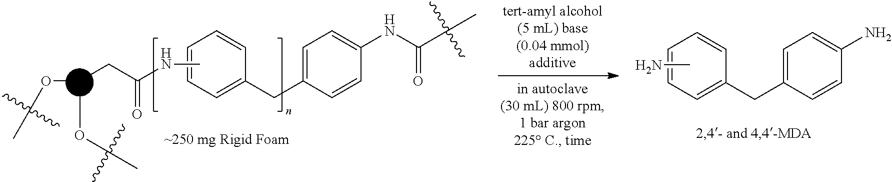

| Entry | Time | Base | Additive | Yield^{[a]} |
|---|---|---|---|---|
| 1 | 2.25 h | KOH | — | 35.0 mg^{b} |
| 2 | 4.5 h | KOH | — | 37.8 mg^{b} |
| 3 | 2.25 h | KOH | 1 mL MeOH^{c} | 17.6 mg |
| 4 | 4.5 h | KOH | 100 μL MeOH | 43.9 mg |
| 5 | 2.25 h | DBTDL | — | 37.4 mg^{b} |
| 6 | 4.5 h | DBTDL | 100 μL MeOH | 47.5 mg |

^{[a]}GC-FID yield of 2,4′- and 4,4′-methylenedianiline determined using 1,3,5-trimethylbenzene as internal standard.
^{b}Precipitate observed in reaction mixture (not further analyzed).
^{c}4 mL tert-amyl alcohol added instead of 5 mL.
DBTDL: dibutyltin dilaurate.

With these conditions in hand, a range of rigid solids and rigid foam PU products were investigated.

### Rigid Foam (Refrigerator)

The rigid foam was taken from a refrigerator door of unknown brand.

The PU was cooled to −196° C. using liquid nitrogen and pulverized to small particles using a mortar and a pestle.

KOH as base. The PU (run 1: 248.7 mg; run 2: 247.1 mg) was deconstructed as described in General Procedure C to afford 2,4′-MDA (run 1: 4.7 mg; run 2: 5.7 mg) as a yellow oil in an average yield of 5.2 mg, 4,4′-MDA (run 1: 43.6 mg; run 2: 46.0 mg) as a yellow solid in an average yield of 44.8 mg, polymeric MDA (run 1: 70.8 mg; run 2: 67.8 mg) as a brown amorphous solid in an average yield of 69.3 mg, and a polyol fraction (run 1: 78.8 mg; run 2: 84.8 mg) as a brown oil in an average yield of 81.8 mg.

DBTDL as base. The PU (run 1: 255.1 mg; run 2: 251.8 mg) was deconstructed as described in General Procedure C to afford MDA (run 1: 53.6 mg; run 2: 52.0 mg) as an orange solid in an average yield of 52.8 mg (2,4′-:4,4′-=1:7.7), polymeric MDA (run 1: 64.3 mg; run 2: 61.5 mg) as a brown amorphous solid in an average yield of 69.3 mg, and a polyol fraction (run 1: 92.6 mg; run 2: 98.3 mg) as a brown oil in an average yield of 95.5 mg.

### Rigid Foam (PIR Insulation)

The rigid foam was taken from an insolation board (Recticel).

The PU was cooled to −196° C. using liquid nitrogen and pulverized to small particles using a mortar and a pestle.

KOH as base. The PU (run 1: 250.8 mg; run 2: 251.6 mg) was deconstructed as described in General Procedure C to afford MDA* (run 1: 50.2 mg; run 2: 53.7 mg) as a yellow oil in an average yield of 52.0 mg (4,4′-:2,4′-; 5.9:1), polymeric MDA (run 1: 54.1 mg; run 2: 57.6 mg) as a brown amorphous solid in an average yield of 55.9 mg, and a “polyol” fraction ** (run 1: 19.4 mg; run 2: 15.0 mg) as a yellow oil in an average yield of 17.2 mg.

*MDA fraction contains aromatic impurities as judged by NMR analysis.

** The “polyol” fractions contains a polar and unknown compound that is not protonated in 1 M HCl (aq.).

### Rigid Foam (Trowel)

The rigid foam was taken from a Trowel acquired in Jem & Fix (Danish hardware store, brand: Eskimo). The PU was shredded using a kitchen grater.

KOH as base. The PU (run 1: 253.5 mg; run 2: 254.8 mg) was deconstructed as described in General Procedure C to afford MDA (run 1: 48.2 mg; run 2: 51.7 mg) as a yellow solid in an average yield of 50.0 mg (4,4′-:2,4′-; 13.0:1), polymeric MDA (n=2; run 1: 29.8 mg; run 2: 23.2 mg) as a yellow solid in an average yield of 26.5 mg, polymeric MDA (n>1; run 1: 32.8 mg; run 2: 27.3 mg) as a brown amorphous solid in an average yield of 30.1 mg, and a polyol fraction (run 1: 85.2 mg; run 2: 76.5 mg) as a brown oil in an average yield of 80.9 mg.

### Solid Thermoplastic (Model)

The PU was formulated with the below indicated reagents:

| Solid Thermoplastic (Model) | wt % |
|---|---|
| Dipropylene glycol | 90 |
| Zeolite | 10 |
| MDI (monomeric mix) | 173 |

The PU was pulverized to fine particles using a mortar and a pestle.

KOH as base. The PU (run 1: 251.0 mg; run 2: 252.5 mg) was deconstructed as described in General Procedure B to afford MDA (run 1: 118.5 mg; run 2: 115.9 mg) as an orange oil in an average yield of 117.2 mg (2,4′-:4:4′-=1.2:1), and a polyol fraction (run 1: 80.0 mg; run 2: 86.4 mg) as a brown oil in an average yield of 83.2 mg.

DBTDL as base. The PU (run 1: 249.7 mg; run 2: 250.6 mg) was deconstructed as described in General Procedure B to afford MDA (run 1: 123.0 mg; run 2: 130.9 mg) as an orange oil in an average yield of 127.0 mg (2,4′-:4:4′-=1.2:1), and a polyol fraction (run 1: 83.9 mg; run 2: 74.5 mg) as a brown oil in an average yield of 79.2 mg.

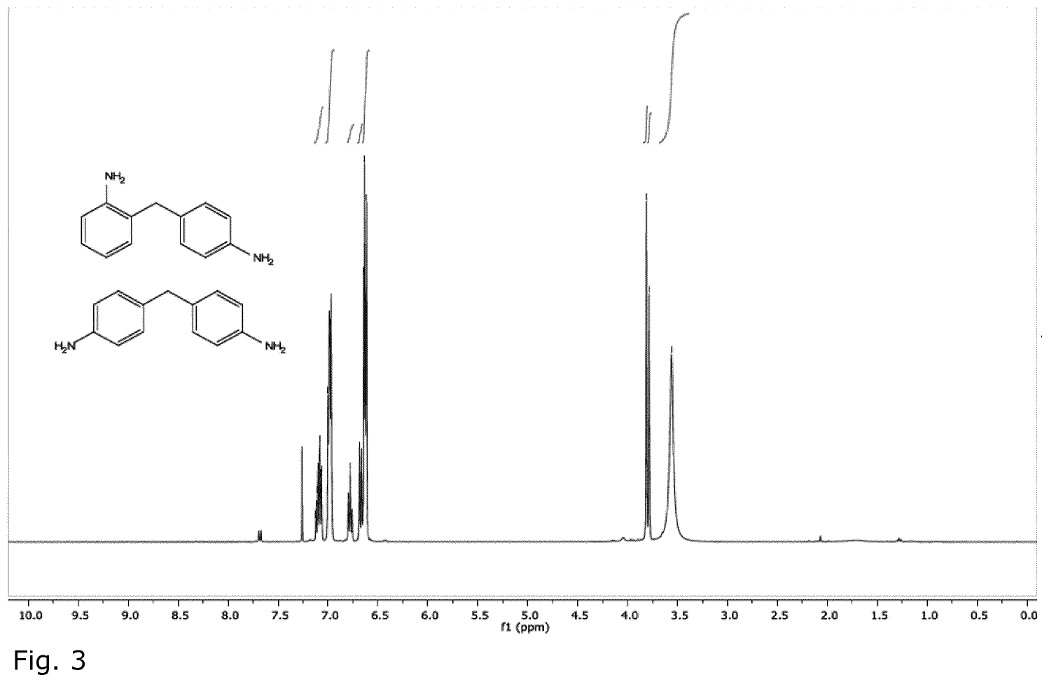

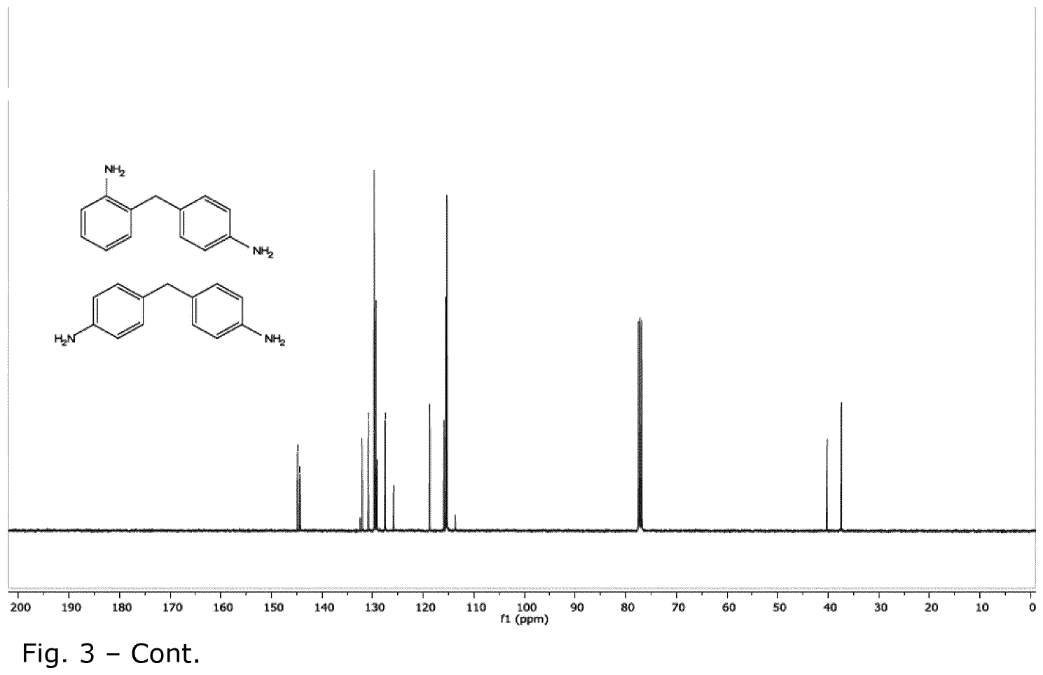

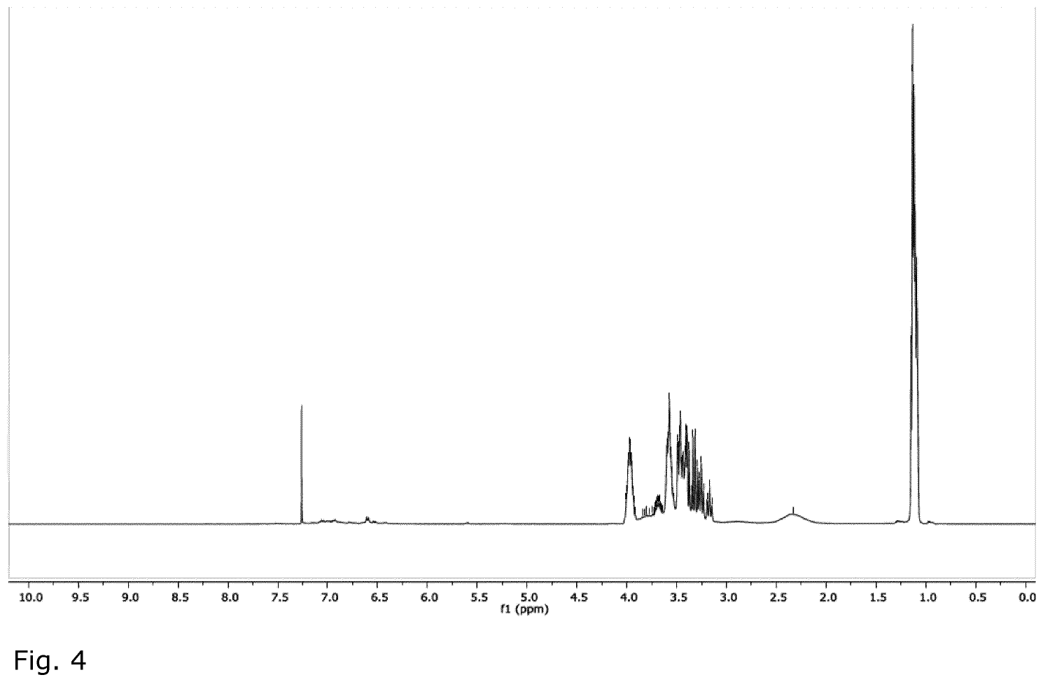

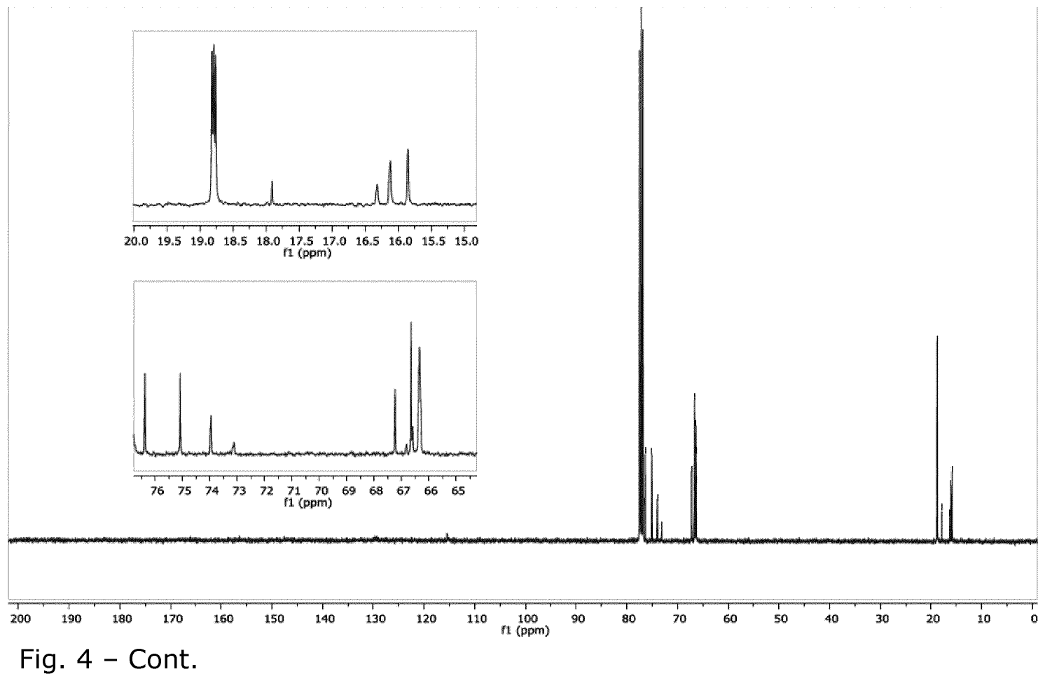

A generic solid thermoplastic was submitted to deconstruction. The thermoplast was disassembled to MDA (see FIG. 3 for NMR spectra) and polyol (see FIG. 4 for NMR spectra) without the need of extended reaction time or addition of methanol to give a mass recovery of 80% or 82% when applying KOH or DBTDL as base, respectively.

### Rigid Foam (End-of-Life Refrigerator)

The end-of-life rigid foam was acquired as big pieces (blended with impurities such as metal and other plastic types) from a scrap facility in which refrigerators are disassembled. The PU was cooled to −196° C. using liquid nitrogen and pulverized to small particles using a mortar and a pestle and visible pieces of copper wire were removed.

KOH as base. The PU (run 1: 250.8 mg; run 2: 250.7 mg) was deconstructed as described in General Procedure C to afford MDA (run 1: 50.5 mg; run 2: 56.0 mg) as a yellow solid with an average yield of 53.3 mg (4,4′-:2,4′-; 10.6:1), polymeric MDA (n=2; run 1: 27.6 mg; run 2: 30.1 mg) as a yellow solid with an average yield of 28.9 mg, polymeric MDA (n>1; run 1: 19.7 mg; run 2: 20.3 mg) as a brown amorphous solid with an average yield of 20.0 mg, and a polyol fraction (run 1: 75.0 mg; run 2: 75.4 mg) as a brown oil with an average yield of 75.2 mg.

### Rigid Solid (Glycerol Model)

The PU was formulated with the below indicated reagents:

| Rigid Solid (Glycerol Model) | wt % |
|---|---|
| Glycerol | 10 |
| Dipropylene glycol | 90 |
| Zeolite | 10 |
| Triethylenediamine | 0.1 |
| MDI (monomeric mix) | 215.8 |

The PU was pulverized to small particles using a mortar and pestle.

KOH as base. The PU (run 1: 257.0 mg; run 2: 252.8 mg) was deconstructed as described in General Procedure C to afford 2,4′-MDA (run 1: 51.7 mg; run 2: 49.9 mg) in an average yield of 50.8 mg, 4,4′-MDA (run 1: 42.4 mg; run 2: 49.2 mg) as a yellow solid in an average yield of 45.8 mg, and a polyol fraction* (run 1: 92. 2 mg; run 2: 92.6 mg) as a yellow oil in an average yield of 92.4 mg.

*The polyol fraction contained aromatic impurities as evident by NMR spectroscopy.

### Rigid Foam (Stucco)

The stucco was ground down using a coffee grinder.

KOH as base. The PU (run 1: 252.0 mg; run 2: 257.2 mg) was deconstructed as described in General Procedure C to afford MDA (run 1: 67.0 mg; run 2: 67.1 mg) as a yellow solid in an average yield of 67.1 mg (4,4′-:2,4′-; 11.8:1), polymeric MDA (n=2; run 1: 18.9 mg; run 2: 19.8 mg) as a yellow solid in an average yield of 19.4 mg, polymeric MDA (n>1; run 1: 7.1 mg; run 2: 16.3 mg) as a brown amorphous solid in an average yield of 11.7 mg, and a polyol fraction (run 1: 142.0 mg; run 2: 105.8 mg) as a brown oil in an average yield of 123.9 mg.

Characterization data for polyMDA (n=2): 1H NMR (400 MHZ, CDCl₃): δ 6.87 (t, J=8.3 Hz, 4H), 6.81-6.75 (m, 2H), 6.55-6.46 (m, 5H), 3.71-3.65 (m, 4H), 3.54-3.23 (m, 6H) ppm. ¹³C NMR (101 MHZ, CDCl₃): δ 144.8, 144.4, 142.8, 132.3, 132.2, 131.5, 129.7, 129.4, 129.4, 127.9, 126.0, 116.2, 115.6, 115.4, 40.4, 37.6 ppm. HRMS (ESI+): m/z calcd. for C₂₀H₂₁N₃ [M+H]⁺: 304.1808, found 304.1802.

### Rigid Foam (Pipe Insulation)

KOH as base. The PU (run 1: 249.3 mg; run 2: 249.6 mg) was deconstructed as described in General Procedure C to afford MDA (run 1: 46.8 mg; run 2: 54.3 mg) as a yellow solid in an average yield of 50.6 mg (4,4′-:2,4′-; 15.3:1), polymeric MDA (n=2; run 1: 22.8 mg; run 2: 21.7 mg) as a yellow solid in an average yield of 22.3 mg, polymeric MDA (n>1; run 1: 27.7 mg; run 2: 31.8 mg) as a brown amorphous solid in an average yield of 29.8 mg, and a polyol fraction (run 1: 81.5 mg; run 2: 78.1 mg) as a brown oil in an average yield of 79.8 mg.

### Flexible Solid (Inline Skating Wheel)

The PU was taken from an end-of-life inline skating wheel. The PU was shredded using a kitchen grater.

KOH as base. The PU (run 1: 252.4 mg; run 2: 257.2 mg) was deconstructed as described in General Procedure C to afford 4,4′-MDA (run 1: 32.9 mg; run 2: 32.2 mg) as a yellow solid in an average yield of 32.6 mg and a polyol fraction (run 1: 172. 3 mg; run 2: 185.6 mg) as a yellow oil in an average yield of 179.0 mg.

### Flexible Solid (Adhesive, Sealant)

“Weicon Urethane 45” was cured according to producer's instructions.

The PU was shredded using a kitchen grater.

KOH as base. The PU (run 1: 251.9 mg; run 2: 258.6 mg) was deconstructed as described in General Procedure C to afford diisopropylnaphtalene (isomeric mixture, run 1: 1.7 mg; run 2: 2.0 mg) as a colorless oil with an average yield of 1.9 mg, 4,4′-methylene-bis-(3-chloro-2,6-diethylaniline) (run 1: 10.7 mg; run 2: 10.3 mg) as a yellow solid with an average yield of 10.5 mg, 2,4-TDA (run 1: 9.4 mg; run 2: 9. 4 mg) as a yellow solid with an average yield of 9.4 mg, a polyol fraction (run 1: 134. 0 mg; run 2: 157.5 mg) as a yellow oil with an average yield of 145.8 mg, and all the combined “waste fractions” * from column chromatography that had low or no UV absorbance at 254 nm (run 1: 41.0 mg; run 2: 41.2 mg) as yellow oil with an average yield of 41.1 mg.

*GC-MS and HRMS data suggest that this fraction could contain diisononyl cyclohexane-1,2-dicarboxylate: HRMS (ESI+): m/z calcd. for C₂₆H4804 [M+H]⁺: 425.3625, found 425.3631.

Diisopropylnaphtalene (isomeric mixture) GC-MS (ESI+): m/z 213.4 ([M+1], 9%), 212.4 ([M], 54%), 198.3 (M+1-CH₃, 16%), 197.3 (M-CH₃, 100%), 181.2, 178.2, 169.3, 165.3, 155.3 (41%), 150.1 (4%), 141.2 (12%), 128.3 (6%), 115.2 (5%), 91.2 (4%), 83.2 (2%), 77.2 (2%), 63.2 (1%).

4,4′-methylene-bis-(3-chloro-2,6-diethylaniline)

¹H NMR (400 MHZ, CDCl₃): δ 6.62 (s, 2H), 4.04 (s, 2H), 3.68 (s, 4H), 2.80 (q, J=7.6 Hz, 4H), 2.43 (q, J=7.5 Hz, 4H), 1.24-1.09 (m, 12H). ¹³C NMR (101 MHZ, CDCl₃): § 140.9 (2C), 132.3 (2C), 127.9 (2C), 127.8 (2C), 126.5 (2C), 126.0 (2C), 37.4, 24.3 (2C), 22.4 (2C), 13.1 (2C), 12.2 (2C). HRMS (ESI+): m/z calcd. for C₂₁H₂₈Cl₂N₂ [M+H]⁺: 379.1702, found 379.1705.

### Flexible Solid (Adhesive, Sealant)

“Weicon Urethane 80” was cured according to producer's instructions.

The PU was shredded using a kitchen grater.

KOH as base. The PU (run 1: 253.0 mg; run 2: 251.3 mg) was deconstructed as described In General Procedure C to afford diisopropylnaphtalene (isomeric mixture, run 1: 23.9 mg; run 2: 24.0 mg) as a colorless oil with an average yield of 24.0 mg, 4,4′-methylene-bis-(3-chloro-2,6-diethylaniline) (run 1: 26.8 mg; run 2: 27.2 mg) as a yellow solid with an average yield of 27.0 mg, 2,4-TDA (run 1: 15. 5 mg; run 2: 19.7 mg) as a yellow solid with an average yield of 17.6 mg, an unknown by-product (run 1: 25.3 mg; run 2: 11.3 mg) as a colorless oil with an average yield of 18.3 mg, a polyol fraction (run 1: 40.1 mg; run 2: 61.5 mg) as a yellow oil with an average yield of 50.8 mg, and all the combined “waste fractions” * from column chromatography that had low or no UV absorbance at 254 nm (only isolated in run 2: 95.3 mg) as yellow oil.

*According to NMR this fraction resembles a polyol fraction.

### Example 3—Deconstruction of PU Textiles

To a PTFE inlay fitted with an oval stir bar was added PU sample (250 mg), KOH (1.9 mg, 0.033 mmol), and tert-amyl alcohol (5 mL) in an argon-filled glovebox. The PTFE inlay was placed in an autoclave reactor, which was sealed and fastened using a hex key. The reactor was placed in a pre-heated aluminum block at 225° C. for 4.5 h at 800 rpm stirring rate. The reactor was then cooled to room temperature. The reactor was opened and the thermowell was washed with EtOAc. The content of the reactor was transferred washing with EtOAc to a round-bottomed flask or a vial in which mesitylene (50 μL) was previously added as an internal standard (exact mass of mesitylene was noted). An aliquot of 250 μL was transferred to a vial and the mixture was quickly filtered through a small cotton and celite plug (3-5 mm of celite in a glass pipette) followed by additional EtOAc into a 2 mL vial suited for GC-MS. The sample was analyzed using the GC method described under Materials and Methods. The GC yield of dianilines was determined using pre-made calibration curves. The aliquot was returned to the main crude mixture and the mixture was purified by automated flash column chromatography using an EtOAc/heptane eluent system from 1:9 to pure EtOAc. Following elution of anilines the polyol fraction was collected by flushing the column with MeOH.

### Fabric (Tights)

The tights (brand: VRS, 10 denier, black, “Løbefri Strømpebukser”, 27% elastane and 73% polyamide) was acquired in a local supermarket.

The tights were cut into smaller pieces with a pair of scissors.

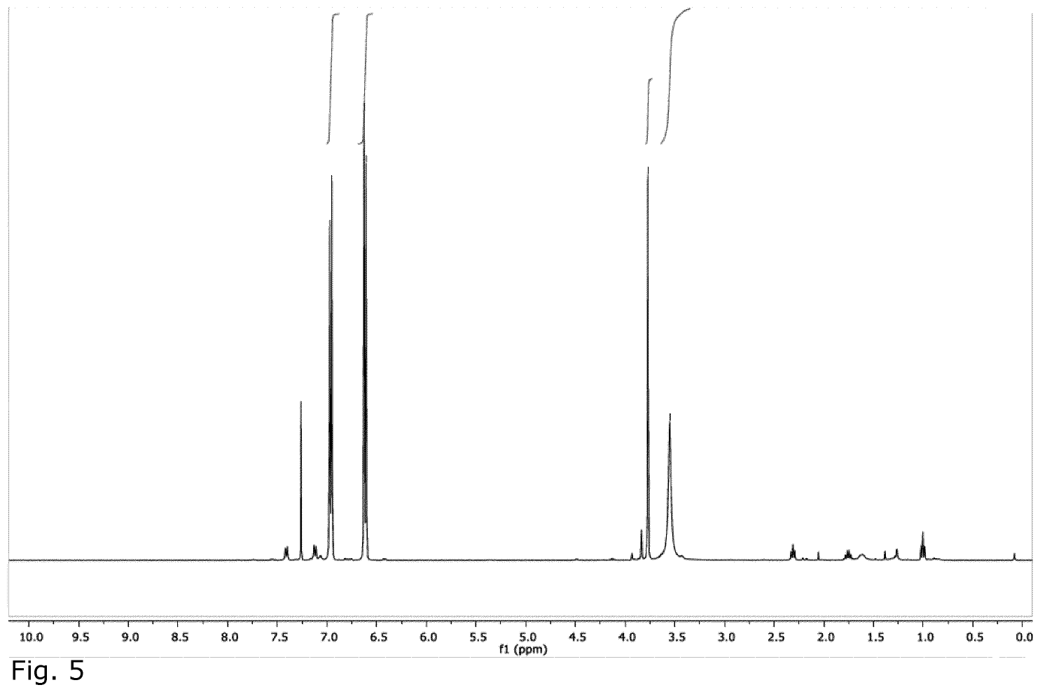

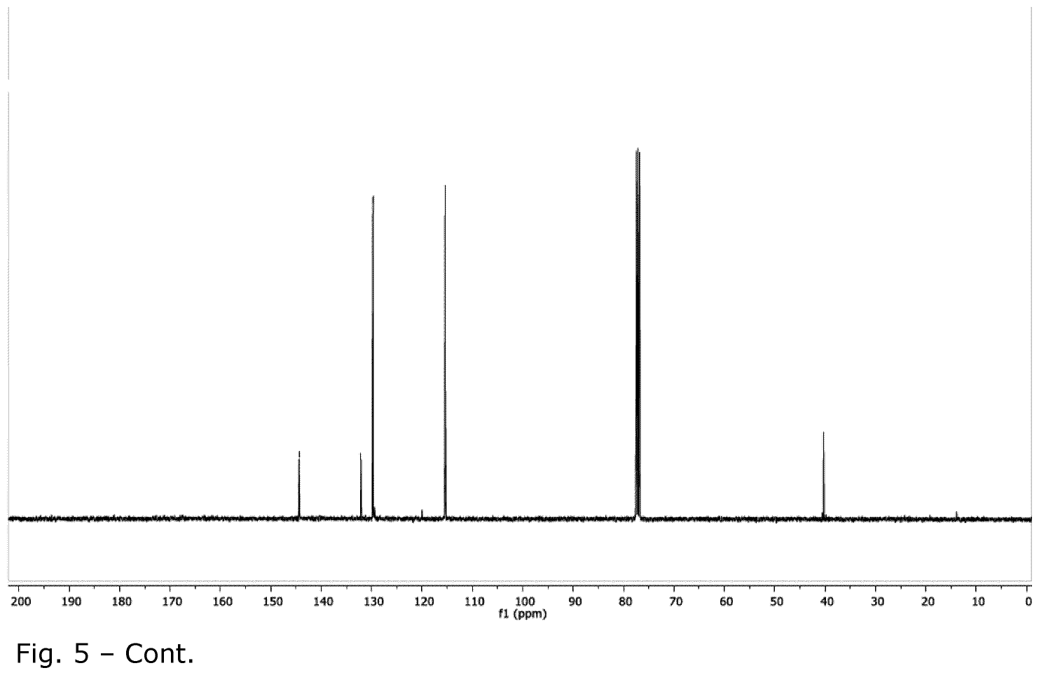

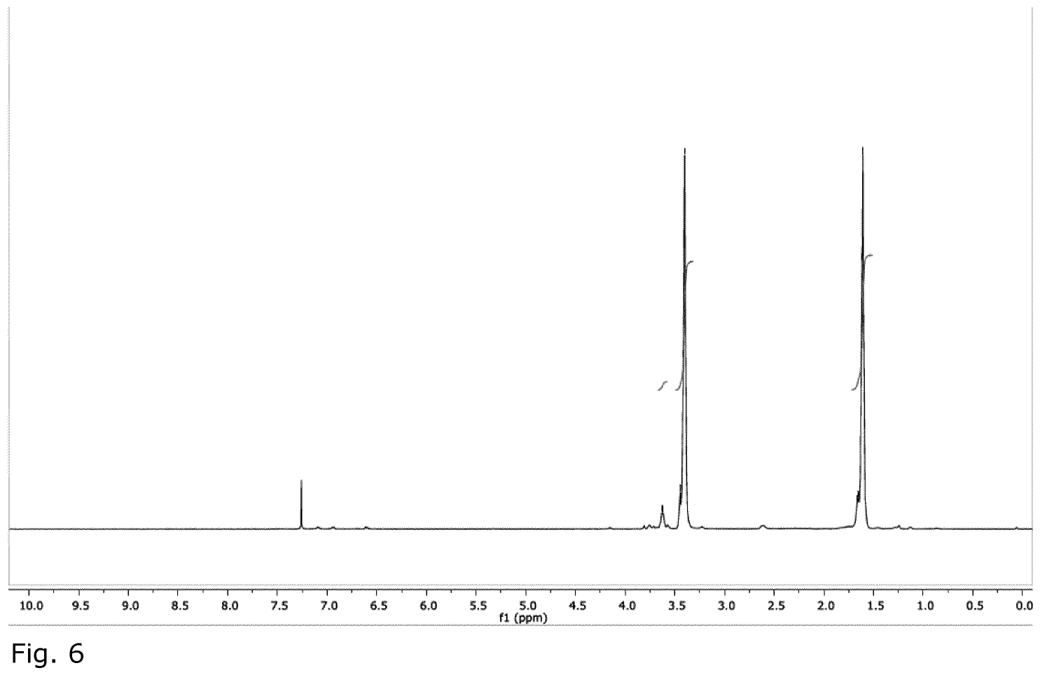

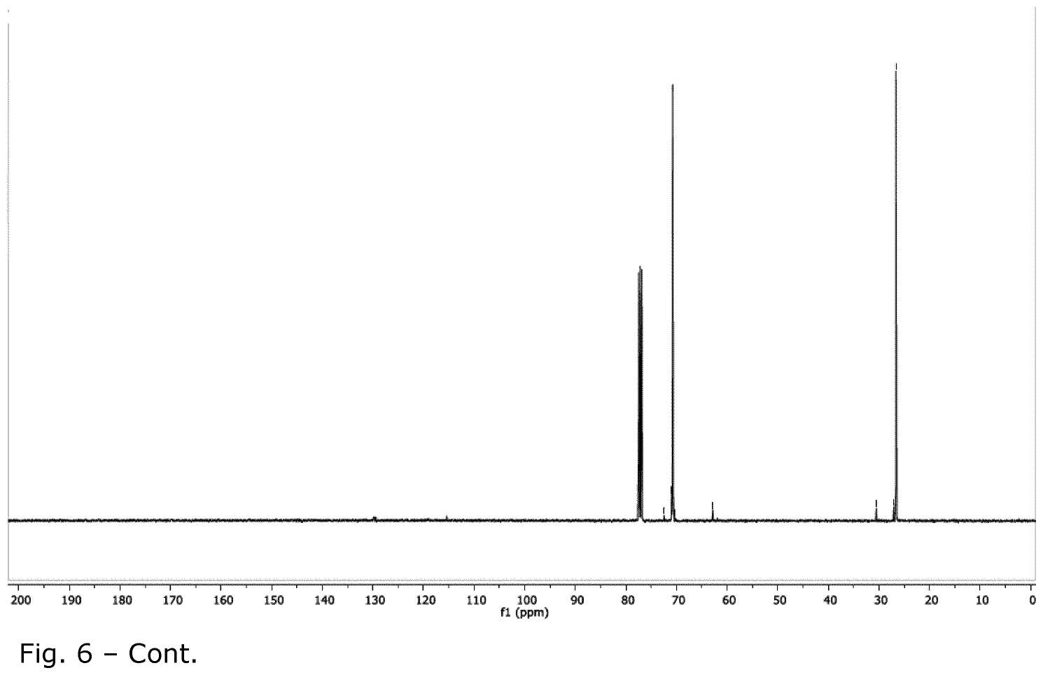

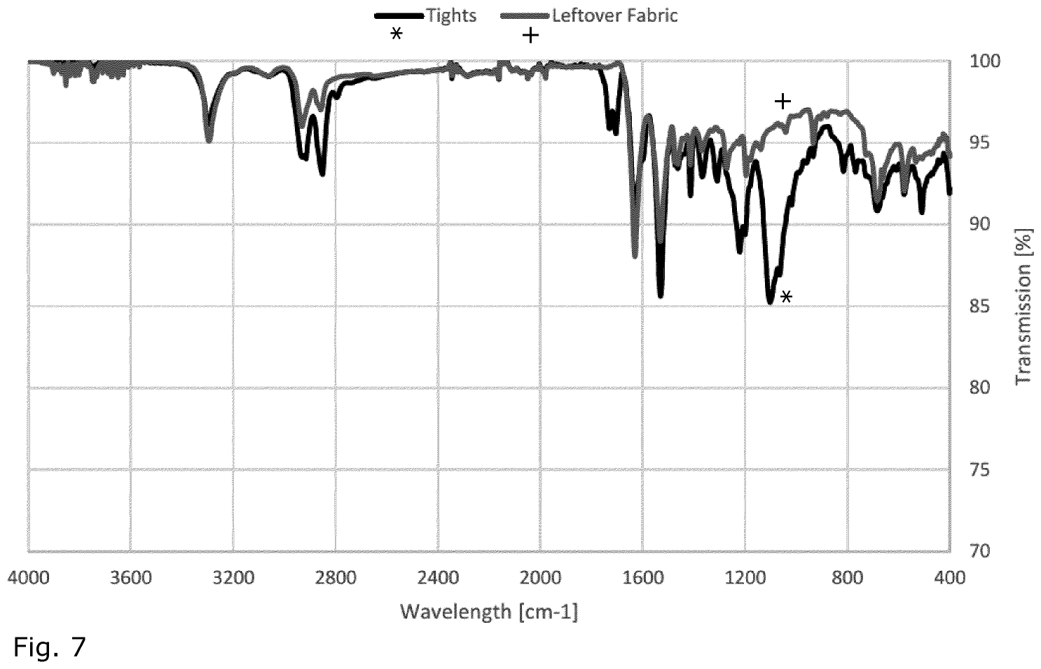

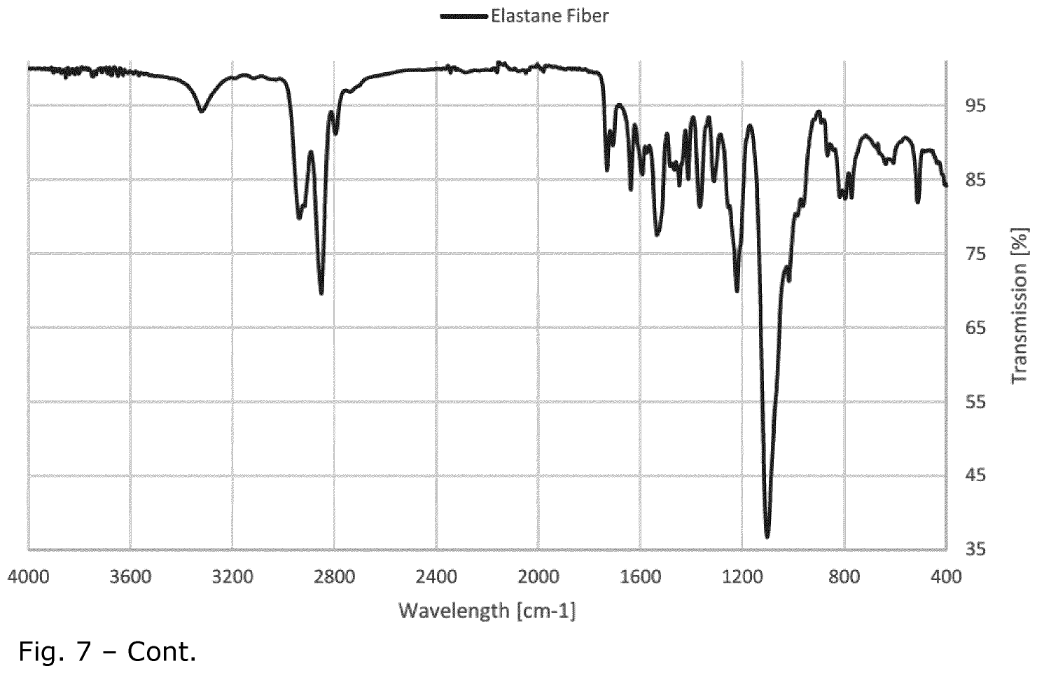

KOH as base. The PU (run 1: 252.5 mg; run 2: 255.2 mg) was deconstructed as described above to afford 4,4′-MDA (run 1: 11.3 mg; run 2: 12.0 mg, FIG. 5) in an average yield of 11.7 mg and a polyol fraction (run 1: 46.4 mg; run 2: 63.0 mg, FIG. 6) as a viscous, yellow oil in an average yield of 54.7 mg, and leftover, black fabric (run 1: 163.7 mg; run 2: 166.9 mg) in an average mass of 165.3 mg. Infrared spectroscopy shows the removal of the polyurethane (elastane) from the textile fabric (FIG. 7).

### Example 4-Scale-Up to 2 G Scale and Precipitation of Amines as Salts

### Glovebox Setup

To a PTFE inlay fitted with a stir bar was added Flexible Foam (Mattress) (2002.0 mg), KOH (15.2 mg, 0.27 mmol), and tert-amyl alcohol (5 mL) in an argon-filled glovebox. The PTFE inlay was placed in an autoclave reactor, which was sealed and fastened using a hex key. Outside the glovebox the reactor was connected to the 5000 Multi Reactor System. The manifold and the reactor were pressurized with argon to 25 bar. The reactor was disconnected from the manifold and the reactor was placed in a pre-heated aluminum block at 225° C. for 6.0 h at 800 rpm stirring rate. The reactor was then cooled to room temperature. The pressure was slowly released. The reactor was opened and the thermowell was washed with EtOAc. The content of the reactor was transferred washing with EtOAc to a round-bottomed flask and concentrated under reduced pressure. An oval stir bar and CPME (20 mL) was added. Under stirring at room temperature 3 M HCl in CPME (2.6 mL, 2.2 equiv.) was added dropwise. The resulting suspension was allowed to stir at room temperature, filtered, and washed with excess CPME. The precipitated dianiline was dried in vacuo to afford 2HCl·TDA (701.9 mg) as a beige solid and the filtrate was concentrated under reduced pressure, dried in vacou to afford polyol (1.3908 g) as a yellow oil.

### Bench Setup

To a PTFE inlay fitted with a stir bar was added Flexible Foam (Mattress) (2001.7 mg), KOH (15.2 mg, 0.27 mmol), and tert-amyl alcohol (5 mL). The PTFE inlay was placed in an autoclave reactor, which was sealed and fastened using a hex key. The reactor was connected to the 5000 Multi Reactor System. The manifold was purged with argon by pressurizing and depressurizing the system. Finally, the reactor was pressurized with argon to 25 bar. The reactor was disconnected from the manifold and the reactor was placed in a pre-heated aluminum block at 225° C. for 6.0 h at 800 rpm stirring rate. The reactor was then cooled to room temperature. The pressure was slowly released. The reactor was opened and the thermowell was washed with EtOAc. The content of the reactor was transferred washing with EtOAc to a round-bottomed flask and concentrated under reduced pressure. An oval stir bar and CPME (20 mL) was added. Under stirring at room temperature 3 M HCl in CPME (2.6 mL, 2.2 equiv.) was added dropwise. The resulting suspension was allowed to stir at room temperature, filtered, and washed with excess CPME. The precipitated dianiline was dried in vacuo to afford 2HCl·TDA (692.0 mg) as a beige solid and the filtrate was concentrated under reduced pressure, dried in vacou to afford polyol (1.3867 g) as a yellow oil.

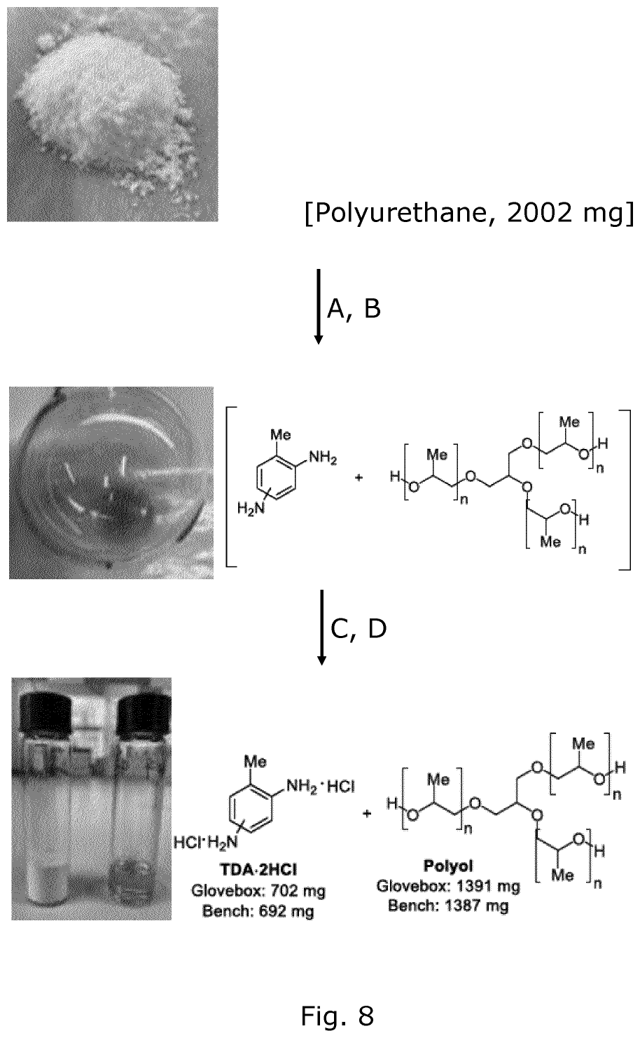

FIG. 8 illustrates the process from starting material to the mixture comprising polyols and amines and subsequent fractions of polyol and precipitated amine salts.

### Example 5—Scale-Up to 50 G Scale

Flexible Foam (Mattress, 50 g), KOH (0.38 g, 6.8 mmol), and tert-amyl alcohol (125 mL) were added to the large stainless steel autoclave. Note: the PU granulate was barely “wetted”. The autoclave was sealed under atmospheric air and heated from room temperature to 225° C. Stirring was initiated as the temperature reached 150° C. The desired temperature was reached after appr. 45 minutes and 6 h reaction time was allowed from this point. The reaction was allowed to cool slowly overnight.

Upon opening of the autoclave significant amounts of gas bubbled off from the brown liquid mixture. The product mixture was filtered and concentrated. The product mixture was diluted with CPME (100 mL) and cooled to 0° C. A solution of HCl (3 M in CPME, 65 mL, 2.2 equiv.) was carefully added (0.5 h) to precipitate the double HCl salt of TDA. The salt was isolated by suction filtration and very thorough washing* with additional CPME. The precipitate and filtrate were dried in vacuo to obtain 2HCl TDA (18.41 g, corresponds to 11.53 g TDA) as a beige solid and polyol (32.78 g) as a yellow oil, respectively, summing up to a total mass recovery of 89%.

Note: 2HCl TDA still contains a minor amount of polyol-more thorough washing, probably with a different solvent, is needed.

*After several washes was the salt transferred to a round-bottomed flask and stirred vigorously with CPME before returning the suspension to the filtration setup.

The internal pressure was recorded using an analog manometer during the reaction:
0 h (225° C. reached): 1-3 bar—no significant reading on pressure gauge
1 h: 10 bar
2 h: 15 bar
4 h: 15-20 bar
6 h: 15-20 bar

After cooling overnight: 1-3 bar—no significant reading on pressure gauge. However, gas release over an extended period of time upon opening was observed (CO₂ in solution at lower temperatures).

After isolation the polyol was kept in a capped round-bottomed flask on the benchtop for 20 days before OH-numbers and water content were determined:

| | OH-number | Water |
|---|---|---|
| | [mg KOH/g] | content [wt %] |
| Recycled polyol, test 1 | 56.7 | 0.108 |
| Recycled polyol, test 2 | 58.5 | 0.112 |
| Recycled polyol, test 3 | 59.0 | 0.106 |
| Average | 58.1 | 0.108 |
| Virgin polyol | 54.5-58.5 | <0.05 |
| specifications | | |

Analysis of the isolated polyol revealed an OH-number (hydroxyl value) of 58.1 (mg KOH)/g within specifications of the original virgin polyol (54.5-58.5 mg KOH/g).

### Example 6—Valorization of Dicarbamate/Amine-Carbamate/Amine Phase from Split-Phase Alcholysis

Initially a flexible foam was subjected to split-phase alcoholysis in accordance with e.g. WO2019219814. Thus, Pentaerythritol (50 wt %, 2.0107 g), 2-pyrrolidone (10 wt %, 404.2 mg), and bismuth neodecanoate (BK850, 1 wt %, 48.1 mg) were added to a 50 mL cylindrical pressure tube (COtube) equipped with an oval stir bar. The pressure tube was sealed with a Teflon seal and stirred at 800 rpm in a pre-heated oil bath at 200° C. Flexible Foam (4.0351 g) was added in portions of approximately 0.67 g with intervals of approximately 23 minutes. After a total dissolution time of 140 minutes, the reaction mixture was allowed to stir at 200° C. for another 180 minutes. The reaction mixture was cooled to room temperature and the phases were allowed to separate over 64 hours. The upper phase (yellow oil, 2.6949 g) was transferred by glass pipette to an 8 mL vial and the lower phase (black solid, 3.0405 g) was transferred to an 8 mL vial by gentle melting and decantation.

The lower phase comprising mainly aromatic dicarbamates, amine-carbamates and amines was further subjected to the process of the present invention. Thus, to a PTFE inlay fitted with a stir bar was added the lower phase from the split-phase glycolysis (run 1: 501.0 mg; run 2: 512.3 mg), KOH (1.9 mg), and tert-amyl alcohol (5 mL) under an atmosphere of air. The PTFE inlay was placed in an autoclave reactor, which was sealed and fastened using a hex key. The reactor was placed in a pre-heated aluminum block at 225° C. for 2.25 h with a 800 rpm stirring rate. The reactor was cooled to room temperature. The reactor was opened and the thermowell was rinsed with a spatula and EtOAc. The content of the reactor was transferred washing with EtOAc to a round-bottomed flask and concentrated under reduced pressure (1H NMR recorded). The crude mixture was purified by automated flash column chromatography using an EtOAc/heptane eluent system from 1:9 to pure EtOAc. Following elution of anilines the polyol fraction was collected by flushing the column with MeOH.

TDA was isolated as a red solid (run 1: 41.1 mg; run 2: 56.5 mg) in an average yield of 48.8 mg (2,4-: 2:6-=5.0:1) and a mixture of mainly pentaerythritol and polyol was isolated as a brown solid (run 1: 253.8 mg; run 2: 343.8 mg) in an average yield of 298.8 mg

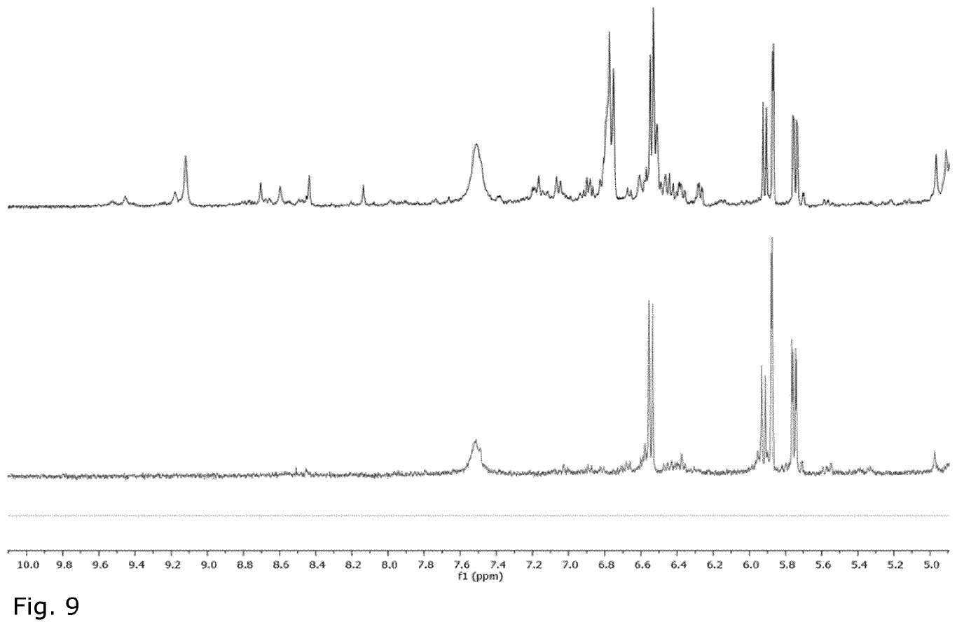

FIG. 9 shows a comparison of the NMR spectrum of the lower phase prior to the method of the present invention (top) and the NMR spectrum of the reaction mixture after alcoholysis of the lower phase according to the present invention (bottom). It is evident that a much more homogenous fraction of anilines is obtained as compared to the lower phase from split phase alcoholysis.

### ITEMS OF THE INVENTION

1. A method for production of a mixture comprising polyol(s) and amine(s) from a polyurethane, said method comprising the steps of:
   a) providing a starting material comprising the polyurethane,
   b) suspending the starting material comprising the polyurethane in a suspending agent comprising an alcohol to provide a polyurethane suspension,
   c) heating the polyurethane suspension to a temperature in the range of 150° C. to 400° C. to provide said mixture comprising polyol(s) and amine(s),
wherein said alcohol is selected from the group consisting of a C₄-C₁₂ tertiary alcohol, and a C₃-C₁₀ secondary alcohol.
2. The method according to item 1, wherein the method comprises an additional step after step c) of
   c′) separating said mixture comprising polyol(s) and amine(s) from said starting material to provide said starting material free of polyurethane.
3. The method according to any one of items 1-2, wherein the method comprises an additional step after step c) of
   d) separating said polyol(s) from said amine(s) to provide a polyol fraction and an amine fraction.
4. The method according to any one of the preceding items, wherein the suspending agent comprises the alcohol and one or more additional solvents.
5. The method according to any one of the preceding items, wherein the alcohol is a C₄-C₁₀ tertiary alcohol, preferably a C₄-C₈ tertiary alcohol.
6. The method according to any one of the preceding items, wherein the molecular weight of the alcohol is in the range of 50 g/mole to 200 g/mole, such as 55 g/mole to 160 g/mole, preferably 60 g/mole to 100 g/mole.
7. The method according to any one of the preceding items, wherein the alcohol is a mono-valent alcohol.
8. The method according to any one of the preceding items, wherein the alcohol is selected from the group consisting of tert-amyl alcohol, 1,1-dimethylallyl alcohol, tert-butanol, iso-propanol, methyl vinyl carbinol, and terpineol.
9. The method according to any one of the preceding items, wherein the alcohol is tert-amyl alcohol.
10. The method according to any one of the preceding items, wherein the starting material comprising a polyurethane is selected from the group consisting of a polyurethane, a poly (urethane-isocyanurate), a cross-linked polyurethane, a block co-polymer comprising polyurethane, a polymer mixture comprising a polyurethane, or any mixture thereof.
11. The method according to any one of the preceding items, wherein a base is further added to the polyurethane suspension.
12. The method according to any one of the preceding items, wherein a further alcohol or an amine is added to the polyurethane suspension, wherein said further alcohol or amine is a primary alcohol or amine.
13. The method according to any one of the preceding items, wherein step c) has a duration in the range of 0.1-10 h, such as 0.5-8 h, such as 1-6 h, such as 1.5-4 h, such as 1.7-3 h, such as preferably 2-6 h.
14. The method according to any one of items 3-13, wherein the polyol fraction has a hydroxyl value which is no more than 10% above the hydroxyl value of the pure monomer, such as no more than 7%, 5%, 3% such as preferably no more than 2% above the hydroxyl value of the pure monomer.
15. Use of an alcohol selected from the group consisting of a C₄-C₁₂ tertiary alcohol and a C₃-C₁₀ secondary alcohol in the conversion of a polyurethane to provide polyol(s) and amine(s).

### REFERENCES

WO2019219814
WO2020260387
US2002010222A1

1. A method for production of a mixture comprising polyol(s) and amine(s) from a polyurethane, said method comprising the steps of:
a) providing a starting material comprising the polyurethane,
b) suspending the starting material comprising the polyurethane in a suspending agent comprising an alcohol to provide a polyurethane suspension,
c) heating the polyurethane suspension to a temperature in the range of 150° C. to 400° C. to provide said mixture comprising polyol(s) and amine(s),
wherein said alcohol is a C₄-C₈ tertiary alcohol.

2. The method according to claim 1, wherein the method further comprises an additional step after step c) of
c′) separating said mixture comprising polyol(s) and amine(s) from said starting material to provide said starting material free of polyurethane.

3. The method according to claim 1, wherein the method further comprises an additional step after step c) of
d) separating said polyol(s) from said amine(s) to provide a polyol fraction and an amine fraction.

4. The method according to claim 1, wherein the suspending agent comprises the alcohol and one or more additional solvents.

5. The method according to claim 1, wherein the molecular weight of the alcohol is in the range of 50 g/mol to 200 g/mol.

6. The method according to claim 1, wherein the alcohol is a mono-valent alcohol.

7. The method according to claim 1, wherein the alcohol is selected from the group consisting of tert-amyl alcohol, 1,1-dimethylallyl alcohol, tert-butanol, iso-propanol, methyl vinyl carbinol, and terpineol.

8. The method according to claim 1, wherein the alcohol is tert-amyl alcohol.

9. The method according to claim 1, wherein the starting material comprising a polyurethane is selected from the group consisting of a polyurethane, a poly(urethane-isocyanurate), a cross-linked polyurethane, a block co-polymer comprising polyurethane, a polymer mixture comprising a polyurethane, and mixtures thereof.

10. The method according to claim 1, wherein prior to step c) a base is further added to the polyurethane suspension.

11. The method according to claim 1, wherein step b) further comprises adding a further alcohol or a further amine to the polyurethane suspension, wherein said further alcohol or further amine is a primary alcohol or primary amine.

12. The method according to claim 1, wherein step c) has a duration in the range of about 0.1-10 h.

13. The method according to claim 3, wherein the polyol fraction has a hydroxyl value which is no more than 10% above the hydroxyl value of the pure monomer.

14. A method for using an alcohol selected from the group consisting of a C₄-C₁₂ tertiary alcohol and a C₃-C₁₀ secondary alcohol in the conversion of a polyurethane to provide polyol(s) and amine(s).

15. The method of claim 13, wherein the polyol fraction has a hydroxyl value which is no more than about 2% above the hydroxyl value of the pure monomer.

16. The method of claim 5, wherein the molecular weight of the alcohol is in the range of about 50 g/mol to about 200 g/mol.

17. The method of claim 12, wherein step c) has a duration in the range of about 2 h to about 6 h.
